(19) United States
(12) Patent Application Publication
(10) Pub. No.: US 2019/0156910 A1
(43) Pub. Date: May 23, 2019

(54) Methods for Adaptive Laboratory Evolution

(71) Applicant: EUROPEAN MOLECULAR BIOLOGY LABORATORY, Heidelberg (DE)

(72) Inventors: Paula Tuulia Jouhten, Heidelberg (DE); Kiran Raosaheb Patil, Heidelberg (DE)

(21) Appl. No.: 16/315,213

(22) PCT Filed: Jul. 6, 2017
(86) PCT No.: PCT/EP2017/066931

§ 371 Date: Jan. 4, 2019

(30) Foreign Application Priority Data
Jul. 6, 2016 (EP) 16178113.3

(51) Int. Cl. G16B 5/00; G01N 33/50; C12P 7/40

(52) U.S. Cl. CPC G16B 5/00; C12P 7/40; G01N 33/5005

(57) ABSTRACT

The invention relates to adaptive laboratory evolution of cells and/or organisms. In particular, the invention relates to a method for adaptive laboratory evolution of cells or organisms in order to generate desired metabolic traits without the need of genetic engineering. In addition, the invention provides a computer program element and a computer readable medium. Further, the invention relates to cells or organisms obtained by the method of the invention.

### FIELD OF THE INVENTION

The invention relates to adaptive laboratory evolution of cells and/or organisms. In particular, the invention relates to a method for adaptive laboratory evolution of cells or organisms in order to generate desired metabolic traits without the need of genetic engineering. In addition, the invention provides a computer program element and a computer readable medium. Further, the invention relates to cells or organisms obtained by the method of the invention.

### BACKGROUND OF THE INVENTION

Adaptive laboratory evolution refers to the culture of cells or organisms under defined conditions leading to adaptive changes that accumulate in populations of cells or (microbial) organisms during selection under specified growth conditions.

Designed modifications of microbial strains have recently achieved increasing interest e.g. in food industry where genetic engineering is often not desired or cannot be applied at all. In addition, even if genetic engineering can be applied, this method is also limited, in particular in the development of complex traits.

So far, it is difficult to predict how the desired traits develop during adaptive laboratory evolution. In particular, the prediction of desired traits under conditions, in which different parameters or components are varied is challenging.

In particular, it is difficult to establish metabolic traits that do not maximize the fitness of the cells. As natural selection favours cells with higher fitness, the selection of conditions for adaptive evolution is non-intuitive, especially when the desired metabolic trait does not maximize the fitness of the cells in the target conditions. The term “target conditions” or “target niche” refers to the chemical environment for the cells or organisms where the desired application, e.g. production ofa flavour compound or wine fermentation, is intended.

Consequently, there is a need for a method capable to design modifications of metabolic traits and/or to develop complex metabolic traits. In particular, there is a need for methods to evaluate the suitability of chemical environments to evolve a desired metabolic trait.

### SUMMARY OF THE INVENTION

It is object of the invention to provide for a method to design chemical environments for adaptive evolution to develop desired metabolic traits.

This object may be solved by the subject-matter according to one of the independent claims. Embodiments of the present invention are described in the dependent claims and with respect to the drawings.

The described embodiments similarly pertain to a method for evaluating the suitability of a chemical environment, a computer program element and a computer readable medium. Synergetic effects may arise from different combinations of the embodiments although they might not be described in detail. It should be noted that in the context of the present invention the terms “flux” and “function of flux” as well as “fluxes” and “functions of fluxes” will be used interchangeably.

Further on, it shall be noted that all embodiments of the present invention concerning a method, might be carried out with the order of the steps as described, nevertheless this has not to be the only and essential order of the steps of the method. All different orders and combinations of the method steps are herewith described.

According to a first aspect of the present invention, a method for evaluating the suitability of a chemical environment to evolve a desired metabolic trait of a cell or organism is provided. The method comprises the simulation of one or several fluxes in a metabolic model, in particular the simulation of one or several functions of fluxes in a metabolic model. Thereby, the metabolic model comprises a stoichiometric representation of biochemical reactions and import and export of extracellular compounds. The metabolic trait comprises a set of targets, the latter being functions of fluxes occurring in the cell or organism.

The desired metabolic trait does not provide a fitness benefit in a target environment where the desired application of the cell or organism is intended.

In particular, the desired metabolic trait is evolved in an evolution chemical environment where it provides a fitness benefit, while in the target environment the desired trait does not provide a fitness benefit. In the target environment, the desired trait is exploited. In the target environment, the desired trait allows the increase or decrease of at least one desired flux and/or avoids the increase or decrease of at least one flux. In one specific embodiment, in the target environment the desired trait allows the increase in the production flux of a least one desired product.

The skilled person understands that the evolution environment and the target environment differ, e.g. the evolution environment and the target environment differ in their composition.

Thus, a specific embodiment refers to a method for evaluating the suitability of a chemical environment to evolve a desired metabolic trait of a cell or organism, wherein the cell or organism is intended to be used in a different chemical environment where the desired metabolic trait does not provide a fitness benefit.

In other words, some embodiments refer to a method for evaluating the suitability of an evolution chemical environment to evolve a desired metabolic trait of a cell or organism, wherein the desired metabolic trait is exploited in a target environment where it does not provide a fitness benefit.

Using the method according to the present invention, a suitable chemical environment can be determined, which can be used to evolve desired metabolic traits of a cell or organism.

According to an embodiment of the invention, a stoichiometric matrix is used in representing the metabolic model. Alternatively, a graph representation of the metabolic model could be provided.

It should be noted that simulation of one or several functions of fluxes in the model in context of the present invention may refer to an optimisation of the one or several functions of fluxes.

A metabolic trait according to the invention is a metabolic characteristic of a cell or organism build by one or a combination of several targets of the corresponding model of the cell or organism. Thus, a metabolic trait is built by at least one target, preferably at least two targets, such as 3, 4, 5, 6, 7, 8, 9, 10 or more targets. A target represents a function of fluxes occurring in the cell or organism.

Typically, a metabolic trait evolves over several generations of the cell or organism. That means that at least two generations, e.g. at least 10, at least 50, at least 100, at least 200, at least 300 or more generations, preferably at least 50 generations, more preferably about 100 generations of the cell or organism are necessary to evolve a metabolic trait. That means that the cells or organisms are cultured for a certain time in the chemical environment, in particular in the evolution chemical environment, for several days, weeks, months or years.

The term “metabolic trait” refers to metabolic features of the cell or organisms, e.g. the capability to produce a certain product under certain conditions, the capability to survive and reproduce under certain conditions, such as certain pH, certain gas levels, or certain nutrients, preferably certain gas levels or certain nutrients.

A chemical environment is considered as suitable to evolve a metabolic trait, if the chemical environment is suitable to establish a metabolic trait. That means, when the population of cells or organisms is maintained, i.e. cultured, in the chemical environment, a part of the population of the cell or organism has established the metabolic trait, e.g. that at least one cell or organisms has established the metabolic trait. Preferably, at least 5%, at least 10%, at least 20%, at least 30%, at least 40%, at least 50%, at least 60%, at least 70%, at least 80%, at least 90% or more of the population have established the metabolic trait.

The term having “established a metabolic trait” means that the metabolic trait will not be lost in the evolution chemical environment and/or in the target chemical environment in the following generations, such as 2, 10, 100, 1000, 5000 or more generations. In particular, this means that in at least 50%, 60%, 70%, 80% or 90% of the cells having the metabolic trait, this trait is not lost in the following generations.

In context of the present invention, it might be desirable to have at least one function of flux in the model being an up-regulation target, wherein an up-regulation target is a function of flux for which an increase due to exposure to the chemical environment is desired.

It is a particular advantage of the present invention that the suitability of a chemical environment to evolve a particular metabolic trait of a cell or organism can be rated/evaluated quantitatively. A desired application of the present invention refers to evolution of a cell or organism in an evolution niche with a suitable chemical environment determined within the method. The evolution in the determined suitable chemical environment enforces or enhances a desired metabolic trait. Subsequently transferring the cell or organism into a target niche, e.g. a production chemical environment, the enforced or enhanced metabolic trait of the cell or organism will give rise to a desired product, such as a flavour compound, e.g. produced by yeast.

According to an embodiment of the invention, simulation of one or several targets relative to a growth is performed. Thereby, the growth is a function of fluxes through a reaction or reactions that generate biomass components or biomass.

“Target reactions” or “targets” are considered relative to a growth in order to set or introduce a scale in context of the method. This allows to compare different environments with each other. For instance, the selection pressure on a reaction, i.e. on the activity of a given enzyme, in an adaptive evolution can be considered to be higher, the higher a target is required per unit of growth. This is because natural selection in adaptive evolution favours fast/high-yield growth. For example, in a first environment an up-regulation target has a high value relative to a growth in a cell. In a second environment, however, the value of the same target is lower relative to the growth of the cell, in both cases assuming growth optimality. For an increased growth rate or growth yield, the value of the target in the case of the first environment has to become higher than in the second case. With a comparison relative to growth in each of the two cases, respectively, the two chemical environments can be compared to each other on a quantitative level.

In one embodiment, at least one up-regulation target is selected from the group consisting of L-threonine aldolase, pyruvate carboxylase, 4-hydroxyphenylpyruvate decarboxylase, 4-hydroxyphenylacetate dehydrogenase, tyrosol dehydrogenase, tyrosol secretion, and 4-hydroxyphenylacetate secretion, and/or wherein at least one down-regulation target is selected from pyruvate decarboxylase and 4-hydroxypyruvate secretion.

In one specific embodiment, the up-regulation targets are L-threonine aldolase, pyruvate carboxylase, 4-hydroxyphenylpyruvate decarboxylase, 4-hydroxyphenylacetate dehydrogenase, tyrosol dehydrogenase, tyrosol secretion, and 4-hydroxyphenylacetate secretion, and the down-regulation targets are pyruvate decarboxylase and 4-hydroxypyruvate secretion.

According to an embodiment of the invention, simulation is performed while the growth is constrained to a fixed value or within a range.

Such constraining of the growth further improves the possibility of comparison of different chemical environments.

According to an embodiment of the invention, simulation is performed while a constraint is set on any uptakes in the model of the cell or organism. Thereby, an uptake is a function of a flux or fluxes through a reaction or reactions representing the uptake of extracellular compounds.

The uptake of the extracellular compounds can be active, e.g. mediated by transporters or vesicles, or passive by diffusion of the compound into the cell. Also encompassed is the interaction of compounds with a receptors or enzymes of the cell or organism that influences a metabolic flux.

According to an embodiment of the invention, simulation is performed while a sum of uptakes is constrained to a fixed value or is constrained within a fixed range.

For instance, the sum of uptakes can be constrained to a fixed value obtained by minimizing the sum of uptakes at a fixed growth. Such setting would refer to a situation of optimal or near optimal conversion to growth.

According to an embodiment of the invention, simulation is performed with a sum of absolute values of uptakes (i.e. Li norm) constrained to an optimal value.

According to an embodiment of the invention, simulation is performed while any of the uptakes are constrained to a fixed value or constrained within a fixed range.

According to an embodiment of the invention, simulation is performed with any of the uptakes constraint to an optimal value. With the term “optimal value” an extreme value may be denoted. These may e.g. be a minimum or maximum value depending on in which direction the reaction is defined in the model.

According to a further embodiment of the present invention, up-regulation targets and down-regulation targets are optimised into opposite directions.

Thereby, an up-regulation target is a target for which an increase is desired and a down-regulation target is a target for which a decrease is desired. An up-regulation target can therefore increase a desired metabolic trait of a cell or organism. On the other hand, down-regulation targets are those targets that can have an undesired, negative or no effect on a desired metabolic trait.

In a simulation of a cell or organism in a particular chemical environment several possible solutions may arise for upregulation and downregulation targets, differing in their performance to enhance the trait. It is the gist of the invention to select for each environment the worst solution(s) to enhance the trait and to use these worst solution(s) for comparing the chemical environments. In order to compare the chemical environments based on their worst performance of enhancing up-regulation target(s) and/or supressing downregulation target(s), the absolute values of up-regulation targets can be minimized and the absolute values of the down-regulation targets can be maximised.

According to an embodiment of the invention, within the simulation absolute flux values are considered and simulation is performed using the absolute flux values.

In a metabolic model, the signs of the fluxes depend on the direction in which the fluxes are defined in the model. A negative flux indicates that this flux goes into the opposite direction as a corresponding flux with positive sign.

According to an embodiment of the invention, the number of targets exceeding or falling below at least one threshold is optimized. This optimisation may help to improve the chemical environment.

According to an embodiment of the invention, the number of targets relative to growth exceeding or falling below at least one threshold is simulated.

The number of targets relative to growth beyond a pre-set threshold can give information on the coverage of the targets by the chemical environment.

The measure “coverage of targets” is capable to indicate how many fluxes are optimized in a superior manner compared to reference conditions. The pre-set threshold can be a function of flux or functions of fluxes in the reference conditions. Further, optionally a threshold is applied determining how much higher or lower the values of the targets should be compared to the reference conditions.

According to an embodiment of the invention, at least one threshold is determined with respect to functions of fluxes in a reference chemical environment.

A reference chemical environment can be, in principal, arbitrarily chosen. It is one objective of the method to determine chemical environments that allow better evolution of targets in a cell or organism than evolution of the targets in the cell or organism would be in the reference environment. For example, a reference chemical environment for yeast would be a grape must medium used for growth of the yeast for wine production.

According to a further embodiment of the invention, within the simulation at least one inhibited flux is defined.

According to an embodiment of the invention, simulation is performed by constraining fluxes through reactions, which are targets of inhibitors, such as substrate analogs or regulatory triggers, such as 2-deoxyglucose, included as components in the chemical environment.

The term “regulatory trigger” refers to a compound that activates or inhibits pathways, in particular metabolic pathways, in a cell or organism, e.g. by transcriptional regulation.

The term “inhibitor” refers to a compound that inhibits a reaction, in particular a metabolic reaction in a cell or organism, e.g. a substrate analog.

Consequently, according to a further embodiment of the invention, simulation can be performed by constraining those fluxes to zero, which are targets of inhibitors or regulatory triggers included as components in the chemical environment.

According to an embodiment, a method comprises the following additional step of determining a numerical score for the chemical environment. Thereby, the score is indicative for a strength of selection pressure on the targets in the chemical environment. Further, the score is indicative for a coverage of the targets by the selection pressure in the chemical environment.

An explicit example for the definition and determination of a numerical score is provided later on below.

According to an embodiment, a method further comprises the step of determination of a numerical score, wherein the score is indicative for a strength of a worst case selection pressure on the targets in the chemical environment and for a worst-case coverage of the targets by the selection pressure in the chemical environment.

According to a further embodiment, a numerical score is defined based on one of the aforementioned embodiments and further takes the number of components in the chemical environment into account.

Possibly, the lower the number of components in the chemical environment, the more cost-efficient the environment can be. Further, it is often not easy to know a priori how different compounds/components are taken up from the chemical environment. This especially applies to the case when several components are provided together. In case of many components it might become less probable that a cell in the chemical environment can take up and use the components in at least close to optimal proportions. In this context, optimal proportions should be understood as the proportions corresponding to optimal growth.

With further reference to a numerical score, in an embodiment of the invention, the score comprises a function of a strength of a selection pressure and a coverage of the targets by the selection pressure as well as a number of components in the chemical environment.

According to an embodiment, a method comprises the following steps: In a first step a first simulation is performed by imposing a plurality of constraints, thereby determining a first simulation result. One of the constraints is understood to set a condition defining a growth to a fixed value or a fixed range. Another constraint sets thermodynamic bounds on the fluxes of the model. Simulation in the first step is performed by optimizing a sum of uptakes. This optimisation is done by minimizing the sum of uptakes of available components of the chemical environment in order to set the optimal conversion of these components to growth of the up taking cell or organism.

In a second method step a second simulation is performed, thereby using or relying on the first simulation result. As a result, a second simulation result is determined. The second simulation comprises setting a constraint for growth of the cell or organism to a fixed value or, alternatively, constraints the growth within a fixed range. Another constraint, which is set within the second simulation, constraints the sum of uptakes considered in the first simulation to the first simulation result. The second simulation result is indicative for the suitability of the chemical environment and promotes a condition identified as optimizing a sum of up-regulation targets and another condition identified as optimizing a sum of down-regulation targets.

According to a further embodiment, in addition to the aforementioned method steps a third method step is comprised relating to a third simulation, thereby determining a third simulation result. Within this third simulation the number of up-regulation targets enhanced with respect to reference up-regulation targets in a reference chemical environment is optimized. Similarly, a number of down-regulation targets suppressed with respect to down-regulation targets in a reference chemical environment is also optimized within the third simulation.

In a further embodiment of the invention, the determination of the numerical score of a chemical environment is formalised in that the score is given by the following value:

$$value = {W}_{c} \cdot \frac{( n - coverage )}{n} + {W}_{s} \cdot \frac{( 1000 \cdot u - strength )}{n} + {W}_{m} \cdot c .$$

The formula relies on a simulation comprising the first, the second and the third simulation steps described above. In the above formula, n denotes the number of targets and coverage refers to the optimal number of targets obtained within the third simulation step. u denotes the number of up-regulation targets, and strength is the sum of the optimised sum of up-regulation targets and the optimised sum of down-regulation targets obtained in the second simulation step. With c the number of components in the chemical environment is indicated. Finally, W_{c}, Wₛ and Wₘ denote mathematical weights. These weights can be set or chosen by a user. For example, the following values may be chosen for W_{c}, Wₛ and Wₘ: 1000, 100, 1. Thereby, assigning the highest weight for the coverage of targets and lowest importance for the number of chemical components in evaluating chemical environments to evolve a trait comprising of the targets. With the choice of specific values for the weights the result from the first, the second and/or the third simulation step—according to the three terms in the sum above—can be given a larger or smaller influence in the numerical score quantified by the numerical value value.

The above formula provides an example for the determination of an actual numerical score for a chemical environment and allows a quantitative comparison of different chemical environments, which can be used in order to evolve a desired metabolic trait of a cell or organism. With reference to the detailed description of embodiments, an explicit and simplified sample calculation is given. From this sample calculation the determination of a numerical score according to the above formula will become apparent.

The method of the present invention provides a score evaluating the suitability of a chemical environment. This score can be used to evaluate and rank different chemical environments.

According to a further embodiment, the score evaluating the suitability of chemical environment is calculated using a function involving strength and/or coverage and/or number of components in the chemical environment. The skilled person will easily adapt this situation without leaving the scope of the invention.

The method of the present invention can be for examples used for fitness scoring in optimization or search algorithms, such as a genetic algorithm or simulated annealing. When a genetic algorithm is used, the chemical environment can for example represent an individual in the genetic algorithm and the presence or absence of compounds of the chemical environment can represent a gene set (sometimes also called traits in the genetic algorithms).

The method of the present invention may also be used to evaluate which natural environments are suitable to evolve a desired metabolic trait. Thus the chemical environment may be a natural environment for a cell or organism. That means that compounds of at least one natural environment that are known or are determined by methods known to the skilled in the art are used for the evaluation of the suitability of the chemical environment. If a natural environment is determined to be suitable to evolve a desired metabolic trait, the organisms can be isolated from the natural environment. The isolated organism can be analysed for the presence of the metabolic trait. Methods for the analysis of the metabolic trait are known to the skilled person and include for example enzymatic tests, metabolite analysis, characterization of the physiology, flux analysis, determining of the proteome or genome of the organism, by methods known to the skilled person (e.g. next generation sequencing).

A metabolic trait may be a metabolic feature of the cell or organism, e.g. the capability to produce a desired product under certain conditions, the capability to survive and reproduce under certain conditions, such as a certain gas levels, the presence or absence of nutrients, or the resistance or sensitivity to a substance. Preferably the metabolic trait allows the cell or organism to produce of at least one desired product by the cell or organism. This means for example that the cell or organism that acquired the metabolic trait is capable to increase the production by 10%, 20%, 30%, 40%, 50%, 60%, 70%, 80%, 90%, 100%, 150%, 200%, 250%, 300% or more of the desired product relative to the amount of desired product in the cell or organism before applying the method of present invention. Also contemplated is that the cell or organism is capable of producing the desired compound after acquiring the metabolic trait while it was not capable of producing the compound before it acquired the metabolic trait. The metabolic trait may be for example also the reduced production of a product. For example, the reduced production of alcohol, such as ethanol, in an alcoholic fermentation process.

The at least one desired product may be any product that can be produced by a cell or organism, in particular a microorganism, for example a polymer, an acid, an alcohol or an ester. The at least one desired product may be a food compound, preferably an aroma compound. An aroma compound may be for example alcohol, aldehyde, ester, fatty acid, ketone, lactone, branched chain amino acid derived aroma or a precursor thereof, an aromatic compound or pyrazine. Alcohols used as aroma compounds may be for example 1,2-butanediol, 2-butanol, 2-3-butanediol, ethanol, 2-ethylbutanol, 2-ethylhexanol, 2-heptanol, hexanol, isobutanol, 2-methylbutanol, 3-methylbutanol, 2-methylpropanol, 2-nonanol, (Z)-1,5-octadien-3-ol, 2-octanol, 1-octen-3-ol, 1-pentanol, phenylethanol, 2-phenylethanol, 1-nonanol. Examples for aldehydes used as aroma compounds are acetaldehyde, decanal, heptanal, (Z)-4-heptenal, hexanal, 2-hexenal, isohexanal, 2-methylbutanal, 3-methylbutanal, 2-methylpropanal, nonanal, (E,E)-2,4-nonadienal, (Z)-2-nonenal, (E)-2-nonenal, octanal, butanal, pentanal, propanal, propenal, thiophen-2-aldehyde. Examples for esters are methyl acetate, ethyl acetate, ethyl butyrate, ethyl hexonate, ethyl isobutonate, ethyl octanoate, ethyl butanoate, isobutyl butanoate, 2-methyl-1-butyl acetate, 3-methyl-1-butyl acetate, 3-octyl acetate, pentyl acetate, phenethyl acetate, ethyl butyrate, propyl butyrate, 2-hydroxyethyl propionate, 2-methyl-2-ethyl-3-hydroxyhexyl propionate, ethyl 2-methylbutanoate, ethyl 3-methylbutanoate. Examples for fatty acids used as aroma compounds are acetate, butyrate, caproate, decanoate, isobutyrate, 2-methylbutyric acid, 3-methylbutyric acid, octanoate, phenylacetate, propionate, valerate. Examples for lactones used as aroma compounds are δ-decalactone, γ-decalactone, γ-butyrolactone, δ-dodecalactone, δ-octalactone, (Z)-6-dodecen-δ-lactone. Examples for ketones used as aroma compounds are acetophenone, acetone, 2,3-butanedione, 2,3-pentandione, 2-butanone, 3-hydroxy-2-butanone, 2-heptanone, 2-hexanone, 3-methyl-2-butanone, 4-methyl-2-pentanone, 2-nonanone, 2-octanone, 1-octen-3-one, 2-pentanone, 3-pentanone, 2-tridecanone, 2-undecanone. Aromatic compounds used as aroma compounds are for example vanillin, benzaldehyde, β-phenethyl alcohol, phenylethyl acetate or trimethylbenzene. Pyrazines used as aroma compounds are for example 2,3-diethyl-5-methylpyrazine, 2-ethyl-3,5-dimethylpyrazine, 2-methoxy-3-isopropylpyrazine. Branched chain amino acid derived aromas are for example methionol, isovalerate, isobutylacetate, isobutanol, isobutanoate, isoamylacetate, isoamyl alcohol, 2-methylbutanol.

In preferred embodiments the aroma compound may be phenylethyl acetate, or branched chain amino acid derived aromas or precursors thereof, such as methionol, isovalerate, isobutylacetate, isobutanol, isobutanoate, isoamylacetate, isoamyl alcohol, 2-methylbutanol.

Therefore, one embodiment refers to the evolution chemical environment to evolve a desired metabolic trait that leads to the increased generation of phenylethly acetate and/or increased generation of aromas derived from branched-chain amino acids or their precursors in a target chemical environment. Accordingly, the invention refers to a method for evaluating the suitability of a chemical environment to evolve the desired metabolic trait that leads to the increased generation of phenylethyl acetate and/or increased generation of aromas derived from branched-chain amino acids or their precursors in a target chemical environment.

In preferred embodiments, the cell or organism is of industrial relevance. Particularly contemplated are microorganisms. The microorganism may be selected from the group of bacteria, yeasts or molds. An example for an industrial relevant bacterium is Escherichia, such as Escherichia coli. The yeast may be for example Saccharomyces, such as Saccharomyces cerevisiae. An example for mold is Aspergillus, such as Aspergillus niger. Examples of microorganisms that are of industrial relevance are Pediococcus pentosaceus, Lactobacillus sp. such as Lactobacillus acidophilus, Lactococcus lactis, Lactobacillus plantarum, Lactobacillus delbrueckii subsp. bulgaricus, Streptococcus thermophiles, Acetobacter sp. such as Acetobacter ghanensis, Acetobacter fabarum, Luyveromyces sp. such as Kluyveromyces marxianus, Kluyveromyces lactis, Kazachstania exigua, Hansenula polymorpha, Ogataea polymorpha, Chartomium thermophilum, Pichia pastoris, Scheffersomyces stipitis, Issatchenkia orientalis, Torulaspora sp. such as Torulaspora delbrueckii, Schizosaccharomyces pombe, Brettanomyces sp. such as Brettanomyces bruxellensis, Zygosaccharomyces bailii, Ceratocystis fimbriata, Neurospora sp., such as Neurospora sitophila, Zygosaccharomyces rouxii, Ceratocystisfimbriata, Lentinus edodes, Neurospora crassa, Phanerochaete sp., Trametes sp., Sporotrichum thermophile, Vibrio costicola, Rhizopus sp. such as Rhizopus rhizopodiformis, Rhizopus oryzae, Rhizopus oligosporus, Rhizomucor pusillus, Penicillium restrictum, Yarrowia lipolytica, Candida rugose, Candida californica, Aspergillus sp., such as Aspergillus niger, Aspergillus oryzae, Aspergillus flavipes, Penicillium sp. such as Penicillium simplicissimum, Penicillium citrinum, Penicillium candidum, Penicillium brevicompactum, Bacillus sp. such as Bacillus subtilis or Bacillus circulans.

In some embodiments, the methods disclosed herein refer to the evolution of a desired trait of yeast, preferably Saccharomyces cerevisiae, more preferably Saccharomyces cerevisiae that allows the production of molecules desired in fermentation, such as wine fermentation or fermentation with lactic acid bacteria.

Typically, the chemical environment is a culture medium, also called growth medium, for the cell or organism. The culture medium may be a complex medium, i.e. a medium that contains a mixture of components that are not defined and/or of which the quantities are not defined, such as wort or grape must. Alternatively the culture medium may be a defined medium, i.e. a medium of which all compounds and their quantities are known. Preferably, the culture medium may be a defined medium. Typically, a chemical environment comprises one or more carbon sources (e.g. glucose, ethanol, glycerol, fructose, xylose, lactose, methanol, methane, CO₂, arabinose, galactose, gluconic acid, glucuronic acid, ribose, cellulose, hemi-cellulose, pectin, lignin, galacturonic acid, succinic acid, fumaric acid, malic acid, pyruvic acid, fatty acids), one or more nitrogen sources (e.g. peptides, urea, amino acids such as glutamate, aspartate, glutamine, valine, phenylalanine, threonine, serine, amines, (NH₄)₂SO₄), trace elements (e.g. FeSO₄, MnCl₂, CoCl₂, CuSO₄, NaMoO₄, H₃BO₃, KI), and vitamins (e.g. pantothenate, thiamine, myo-inositol, nicotinic acid, pyridoxine, biotin, para-amino benzoic acid). The components in a chemical environment may be introduced in different ionic state or different hydrated forms or in different salt forms. The components in a chemical environment may be introduced in different proportions. Carbon source can provide both carbon for biosynthesis and carbon for the generation of energy or there may be separate substrates for biosynthesis and energy. A chemical environment could also consist of several carbon and nitrogen sources or a single or many substrates being both a carbon and nitrogen sources. The chemical environment may comprise different compounds that are useful for evolving a metabolic trait. An exemplary chemical environment may comprise glycerol, an alcohol, as a major carbon source, and L-valine, an amino acid, as a nitrogen source, in addition to several trace elements and vitamins. An exemplary chemical environment may comprise glucose, (NH₄)₂SO₄, KH₂PO₄, Mg₂SO₄, L-methionine, FeSO₄.7H₂O, ZnSO4.7H₂O, CaCl₂).6H₂O, MnCl₂.2H₂O, CoCl₂.6H₂O, CuSO₄.5H₂O, NaMoO₄.2H₂O, H₃BO₃, KI and Na₂EDTA.2H₂O, d-biotin, para-amino benzoic acid, nicotinic acid, Ca-pantothenate, pyridoxine HCL, thiamine HCl and myo-inositol. The chemical environment may further comprise inhibitors such as 1,5-gluconolactone, 2-deoxyglucose, 2-phosphoglycolate, diamide, D-threose 2,4-diphosphate, sodium iodoacetate, 2-phosphoglycolohydroxamate, 4-chloromercuribenzoic acid, 4-Methylpyrazole hydrochloride, 5-phosphoarabinonate, 6-azauracil, 6-azauridine, acetazolamide, acetoacetone, acetopyruvate, acetoacetyl-CoA, allopurinol, aminoethylpyruvate, antimycin A, ascosteroside, echinocandin B der., ergokonin A, aspartate semialdehyde, aureobasidin A, bafilomycin A1, concanamycin A, hygrolidin, lencanicidin, beta-chloro-L-alanine hydrochloride, carmustine, tetraethylthiuram disulfide, thiuram, caspofungin, echinocandin C der., cerulenin C75, clotrimazole, fenpropimorph, cyproconazole, fluconazole, tubulazole, voriconazole, diethylenetriamine pentaacetic acid, eflornithine, etidronate disodium hydrate, pamidronate disodium salt hydrate, fluorouracil, fluvastatin, lescol, glucosamine, hydroxycarbamide, iodoacetamide, leflunomide, methionine sulfoxime phosphate, methotrexate, methylamine, methylmercury, mycophenolate mofetil, mycophenolic acid, myriocin der., myxothiazol A, strobilurin B, niclosamide, nitisinone, nitrate, N-phosphonoacetyl-L-aspartate, oxalate, P1,P5-Di(adenosine-5′)pentaphosphate(Ap5A), pyridoxylalanine, quercetin, rapamycin, sinefungin, soraphen A, TOFA, squalestatin, squalestatin der., zaragozic acid, terbinafine hydrochloride, terbinafine, naftifine, Triacsin C, triclosan, tritoqualine, valproic acid, and vigabatrin.

The target chemical environment is the environment used to culture the cell or organism to exploit the desired metabolic trait. That means that in the target chemical environment the cell or organism is cultured for example to produce a desired product.

In some embodiments, the chemical environment is an evolution chemical environment used to evolve the metabolic trait of the cell or organism. The chemical environment differs from a target chemical environment used for culturing the cell or organism to exploit the metabolic trait, for example to produce at least one desired product. This allows to evolve traits that are not improving the fitness of the cell or organism in the target environment. For example, if, due to the desired metabolic trait (e.g. the production of a desired product) the growth of the cell or organism is reduced in the target chemical environment, the evolution of the metabolic trait in the target chemical environment, in which the metabolic trait is not a fitness advantage, is hardly feasible or not possible at all. Therefore, the cell or organism is first grown in the evolution chemical environment in which at least one of the targets of the metabolic trait provides a fitness advantage. In this case, the method of the invention aims at the evaluation of the suitability of the evolution chemical environment to evolve a metabolic trait.

The terms “fitness advantage”, “fitness benefit” as used herein mean that the cell or organism having a specific trait has a fitness benefit over another cell or organism not having the specific trait, e.g. that its growth is enhanced compared to another cell or organism not having the specific trait. As already disclosed herein elsewhere the specific trait is established in an evolution environment in which the specific trait conveys a fitness benefit. In the target environment, where the cell or organism is intended to be used, the specific trait does not provide a fitness benefit, i.e. is not growth beneficial. In the target environment, the specific trait is exploited. In some embodiment, in the target environment, the specific trait allows the (enhanced) production of desired molecules and/or allows avoiding or reducing the production of undesired molecules.

In an alternative embodiment, the cell may be a tumour cell or a group of tumour cells or a pathogen.

The method of the invention may also be used for the destabilization of the cell or organism. Particularly, the destabilization of a tumour cell or a pathogen is contemplated. Preferably, the destabilization of a tumour cell is contemplated. This means in the context of the invention that the chemical environment would lead to the evolution of metabolic traits that destabilize the cell or organism, e.g. a tumour cell or a pathogen. The destabilization of the cell or organism may be for example a decreased growth and/or death of the cell or organism.

This means that the chemical environment evaluated by the method of the invention might lead to the evolution of a destabilized cell or organism. This means that the destabilized cell or organism is achieved after at least one generation, typically, after several generations of the cell or organism, such as at least 2, at least 3, at least 4, at least 5, at least 10, at least 20, at least 50 or more generations. Preferably, a metabolic trait that destabilizes the cell is achieved after at most 10000 generations, at most 1000 generations, at most 500 generations, at most 300 generations, at most 200 generations, at most 100 generations, at most 50 generations, at most 30 generations, at most 20 generations, at most 10 generations, at most 5 generations, at most 3 generations.

In embodiments, that aim at the destabilization of a cell, different compound compositions (i.e. chemical environments) may be evaluated for the suitability to evolve a metabolic trait that destabilizes the tumour cell, when the composition is administered to a patient suffering from cancer. Thus, the invention also refers to a composition for use as a medicament. In particular, the invention refers a composition for use in treating cancer.

Another aspect of the invention refers to a method for determining a chemical environment to evolve a metabolic trait of a cell or organism comprising the following steps:
(a) Evaluating the suitability of at least two chemical environments using the method as described herein,
(b) Selecting at least one environment with a suitability to evolve a metabolic trait, which exceeds a threshold of suitability.

As stated above, typically the cell or organism is intended to be used in a different environment (target environment) different from the environment in which the metabolic trait is evolved (evolution environment), wherein in the target environment the metabolic trait does not provide a fitness benefit.

The invention further relates to a method for determining a chemical environment to evolve a metabolic trait of a cell or organism comprising the following steps:
(a) Evaluating the suitability of at least two chemical environments using the method as described herein,
(b) Selecting at least one chemical environment with a suitability to evolve a metabolic trait, which belongs to the chemical environments with the highest potential to evolve a metabolic trait.

In step (b) a chemical environment may be selected which belongs to the 90%, 80%, 70%, 60%, 50%, 40% 30% 20%, 15%, 10%, 5%, 3%, 2% of chemical environments with the highest potential to evolve a metabolic trait of all tested chemical environments. The skilled person understands that also several chemical environments can be selected and used for the evolution of the metabolic trait in parallel.

In a specific embodiment the method providing a chemical environment to evolve a metabolic trait of a cell or organism comprising the following steps:
(a) Evaluating the suitability of at least two chemical environments using the method as described herein,
(b) Selecting at least one chemical environment with a suitability to evolve a metabolic trait, which exceeds a threshold of suitability,
(c) Preparing a chemical environment of step (b).

Another aspect refers to a chemical environment obtainable or obtained by the method described above.

The threshold of suitability may be a pre-set threshold. The threshold of suitability may be set so that the chemical environments exceeding the threshold are the 60%, the 50%, the 40%, the 30%, the 20%, the 10% with the highest potential to evolve a metabolic trait of all tested chemical environments. The skilled person understands that depending on the way of calculation of the suitability of environments, the potential environments to evolve a metabolic trait may also be below a threshold. In a specific embodiment, the environment with the highest potential to evolve a metabolic trait is selected. In another specific embodiment one of the two environments, one of the 3 environments, one of the 4 environments, one of the 5 environments, one of the 6 environments, one of the 10 environments, one of the 20 environments, one of the 50 environments, one of the 100 environments, one of the 500 environments, one of the 1000 environments or more with the highest potential to evolve a metabolic trait is selected.

Moreover, the application is concerned with a method for evolving a metabolic trait of the cell or organism comprising the steps: The invention further relates to a method for determining a chemical environment to evolve a metabolic trait of a cell or organism comprising the following steps:
(a) Evaluating the suitability of at least two chemical environments using the method as described herein,
(b) Selecting at least one chemical environment with a suitability to evolve a metabolic trait, which belongs to the chemical environments with the highest potential to evolve a metabolic trait;
(c) Growing the cell or organism in the chemical environment selected in step (b).

In a specific embodiment, the method for evolving a metabolic trait of the cell or organism comprises the steps,
(a) Evaluating the suitability of at least two evolution chemical environments using the method as described herein,
(b) Selecting at least one chemical environment with a suitability to evolve a metabolic trait, which belongs to the chemical environments with the highest potential to evolve a metabolic trait;
(c) Growing the cell or organism in the selected evolution chemical environment to evolve the desired trait,
(d) Growing the cell or organism in a target chemical environment to produce at least one desired product by the cell or organism.

In addition, the application is concerned with a method for evolving a metabolic trait of the cell or organism comprising the steps:
(a) Evaluating the suitability of at least two chemical environments using the method as described herein,
(b) Selecting at least one environment with a suitability to evolve a metabolic trait, which exceeds a threshold of suitability,
(c) Growing the cell or organism in the chemical environment selected in step (b).

Further, the present application refers to a cell or organism obtained by the methods described herein. In particular, the present application contemplates a cell or organism obtained by a method comprising the following steps:
(a) Evaluating the suitability of at least two chemical environments using the method as described herein,
(b) Selecting at least one environment with a suitability to evolve a metabolic trait, which exceeds a threshold of suitability,
(c) Growing the cell or organism in the chemical environment selected in step (b).

In a specific embodiment, the method for evolving a metabolic trait of the cell or organism comprises the steps,
(a) Evaluating the suitability of at least two evolution chemical environments using the method as described herein,
(b) Selecting at least one evolution chemical environment with a suitability to evolve a metabolic trait, which exceeds a threshold of suitability
(c) Growing the cell or organism in the selected evolution chemical environment to evolve the desired trait,
(d) Growing the cell or organism in a target chemical environment to produce at least one desired product by the cell or organism.

According to a further aspect of the invention, a computer program element is provided, which, when it is executed by a processor, is adapted to carry out any or any combination of the method steps described above.

According to another aspect of the invention, a computer readable medium is provided, which comprises the above computer program element.

It may be seen as a gist of the invention to quantify the suitability of a chemical environment to evolve a desired metabolic trait of a cell or organism. With the described method steps, a quantitative comparison of different chemical environments can be performed in order to determine such chemical environments that allow desired evolution of a cell or organism.

It has to be noted that some of the embodiments of the invention are described with reference to different subject-matters. In particular, some embodiments are described with reference to method type claims whereas other embodiments are described with reference to apparatus type claims. However, a person skilled in the art will gather from the above and the following description that unless other notified in addition to any combination of features belonging to one type of subject-matter also any combination between features relating to different subject-matters is considered to be disclosed with this application.

The aspects and embodiments defined above and further aspects, embodiments, features and advantages of the present invention can also be derived from the examples of embodiments to be described hereinafter and are explained with reference to examples of embodiments. The invention will be described in more detail hereinafter with reference to examples of embodiments but to which the invention is not limited.

A further aspect of the invention refers to a method for determining a selection chemical environment for screening cells or organisms having a desired metabolic trait comprising the following steps:
(a) Evaluating the suitability of at least two chemical environments to evolve a metabolic trait using the method as disclosed herein,
(b) Selecting at least one chemical environment with a suitability to evolve a metabolic trait, which belongs to the chemical environments with the highest potential to evolve a metabolic trait;
   wherein the suitability of the chemical environment to evolve a metabolic trait correlates with the suitability of the chemical environment for screening cells or organisms having a desired metabolic trait.

The suitability of the chemical environment for screening cells or organisms having a desired metabolic trait correlating with the suitability of the chemical environment to evolve a metabolic trait means that a chemical environment that is suitable to evolve a metabolic trait is also suitable for screening cells or organisms having a desired metabolic trait.

A further embodiment refers to a method for screening cells or organisms having a desired metabolic trait using a selection chemical environment,
(a) Determining a selection chemical as defined above,
(b) Growing at least two cells or organisms in the selection chemical environment,
(c) Selecting at least one cell or organism that shows higher growth than one or more of the other cells or organisms in the selection environment; wherein higher growth in the selection environment indicates that the cell or organism contains the desired metabolic trait;

Since the desired metabolic trait provides a fitness benefit in the selection environment, the cell or organism containing the metabolic trait would show higher growth compared to other cells or organisms not containing the desired metabolic trait. Hence, the higher growth of a cell or organism in the selection environment would indicate that the cell or organism contains the desired trait.

“Higher growth” in the context of the invention means the growth of the organism or cell is more than 5%, 10%, 15%, 20%, 30%, 40%, 50%, 100%, 200% or more compared to other cells or organisms grown in the selection environment. The skilled person is aware of methods to determine the growth of a cell or organism. The growth can be e.g. determined by measuring the OD of a solution containing the cell or organism.

The at least two cells or organisms may be for example a cell collection or organism strain collection, such as a yeast strain collection.

Thus, in some embodiments, the term “cell” refers also to “cell line” and the term “organism” refers to “organism strain”.

Hence, in some embodiments, the growth of the organism strain is compared to the growth of the other organism strains in the organism strain collection. In other embodiments, the growth of the cell line is compared to the growth of the other cell lines in the cell line collection. In one embodiment, the one or more of the other cells or organisms is a control cell or control organism of which it is known that it does not contain the desired metabolic trait.

### BRIEF DESCRIPTION OF THE DRAWINGS

FIG. 1 shows a simplified metabolic network and its evolution in an evolution niche (i.e. evolution chemical environment) and a target niche (i.e. target chemical environment).

FIG. 2 shows an illustration of a toy model with exchange reactions for extracellular compounds.

FIG. 3 is an illustration of a genetic algorithm relying on the method of the present invention

FIG. 4A. Histogram of EvolveX calculated combined scores of selection pressure, target coverage, and the number of components in the Evolution niche, relative to the minimum score observed, for the suitability of the particular Evolution niche to evolve an increased generation of branched chain amino acids derived aromas.

FIG. 4B. Histogram of EvolveX calculated combined scores of selection pressure, target coverage, and the number of components in the Evolution niche, relative to the minimum score observed, for the suitability of the particular Evolution niche to evolve an increased generation of Phenylethyl acetate.

FIG. 5. a) L-Phenylalanine and branched chain amino acids are synthesized from distinct precursors in central carbon metabolism. b) Branched chain amino acid derived aromatic compounds are more pronounced in E-strains isolated from lineages evolved on EvolveX designed medium for increased generation of aromas derived from branched chain amino acids (i.e. Ethanol, Glycine L-Arginine as sole carbon and nitrogen sources) than in G-strains isolated from lineages evolved on EvolveX designed medium for increased generation of Phenylethyl acetate (i.e. Glycerol, L-Phenylalanine, L-Threonine) compared to the parental strain. Log 2 fold changes are calculated for triplicate wine must fermentations of the evolved strains compared to triplicate wine must fermentations of the parental strain. The log 2 fold changes marked with a star were found significant (i.e. Dunnett test, >control, p<0.05). c) Increased generation of Phenylethyl acetate was observed in a strain isolated from a lineage evolved on EvolveX designed medium for increased generation of Phenylethyl acetate (i.e. Glycerol, L-Phenylalanine, L-Threonine) compared to the parental strain. d) Ehrlich pathway is the catabolic pathway through which branched chain and aromatic amino acid derived aromas are generated. Ehrlich pathway generates fusel acids and, more abundantly, fusel alcohols that can further react (i.e. esterification) with acids resulting in esters that are important aroma compounds in fermentation products.

FIG. 6A. Histogram of EvolveX calculated combined scores of selection pressure, target coverage, and the number of components in the Evolution niche, relative to the minimum score observed, for the suitability of the particular Evolution niche to evolve S. cerevisiae to enhance the support of lactic acid bacteria (LAB) growth.

FIG. 6B. Evolved strains show improved enhancement of lactic acid bacterial growth. Four-day growth of L. lactis (IL1403) and L. plantarum (WCSFI) inoculated in conditioned-medium (to an initial OD₆₀₀ of 0.01) from the parental S. cerevisiae strain (T8), and the evolved S. cerevisiae strains (A11, A12, B11) normalized by the OD₆₀₀'s of the corresponding yeast strains at the time of collecting the conditioned-medium.

### DETAILED DESCRIPTION OF EMBODIMENTS

Similar or relating components in the several figures are provided with the same reference numerals. The view in the figure is schematic and not fully scaled.

In the following, a simple toy model example for an algorithm based on the method of the invention is presented. The toy model is meant to provide a simple and comprehensible example. Neither the invention nor one of the embodiments described above is limited to the simple scenario described with respect to the toy model.

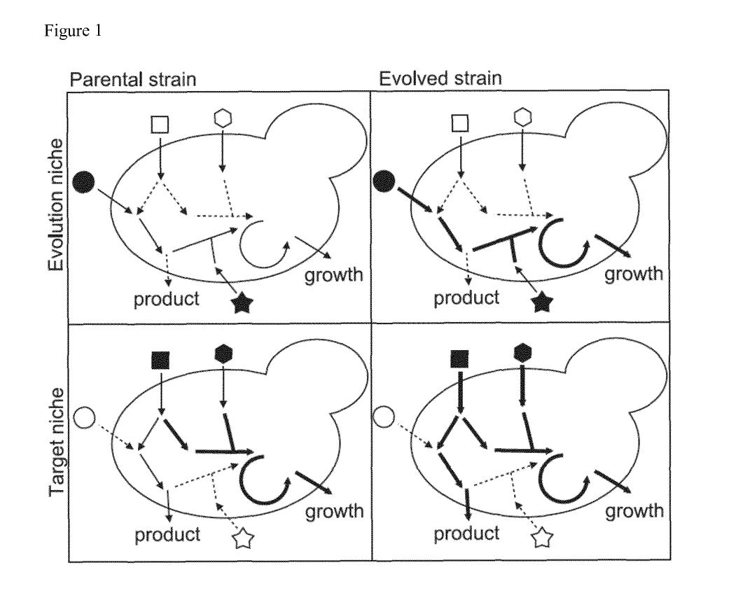

The illustration in FIG. 1 describes with a simplified metabolic network the separation of evolution and target niches, wherein niche is a chemical environment, and thereby the evolution of a metabolic trait that does not provide a fitness benefit in target niche.

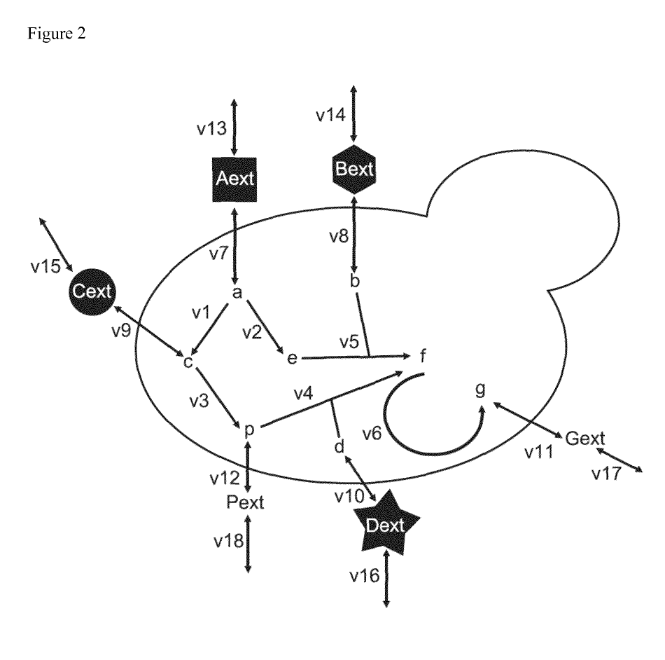

The simplified metabolic network used in the illustration in FIG. 1 is next developed into a metabolic model, which is the toy model considered here used to present an example of calculations based on the method steps of the invention. In FIG. 2 the metabolic network is augmented with exchange reactions for extracellular compounds, referring to import and export of extracellular compounds. Further, the fluxes and metabolites are indicated with identifiers in FIG. 2.

From the reactions indicated in FIG. 2, the following stoichiometric matrix S of the model can be deduced and the following vectors—mass balance vector (b-vector), lower- and upper-bound vectors (lb- and ub-vectors), and biological objective vector (c-vector) are introduced:

$$S \text{-} matrix ( the stoichiometric matrix ) = \begin{matrix} & v 1 & v 2 & v 3 & v 4 & v 5 & v 6 & v 7 & v 8 & v 9 & v 10 & v 11 & v 12 & v 13 & v 14 & v 15 & v 16 & v 17 & v 18 \\ a & - 1 & - 1 & 0 & 0 & 0 & 0 & - 1 & 0 & 0 & 0 & 0 & 0 & 0 & 0 & 0 & 0 & 0 & 0 \\ b & 0 & 0 & 0 & 0 & - 1 & 0 & 0 & - 1 & 0 & 0 & 0 & 0 & 0 & 0 & 0 & 0 & 0 & 0 \\ c & 1 & 0 & - 1 & 0 & 0 & 0 & 0 & 0 & - 1 & 0 & 0 & 0 & 0 & 0 & 0 & 0 & 0 & 0 \\ d & 0 & 0 & 0 & - 1 & 0 & 0 & 0 & 0 & 0 & - 1 & 0 & 0 & 0 & 0 & 0 & 0 & 0 & 0 \\ e & 0 & 1 & 0 & 0 & - 1 & 0 & 0 & 0 & 0 & 0 & 0 & 0 & 0 & 0 & 0 & 0 & 0 & 0 \\ f & 0 & 0 & 0 & 1 & 1 & - 1 & 0 & 0 & 0 & 0 & 0 & 0 & 0 & 0 & 0 & 0 & 0 & 0 \\ g & 0 & 0 & 0 & 0 & 0 & 1 & 0 & 0 & 0 & 0 & - 1 & 0 & 0 & 0 & 0 & 0 & 0 & 0 \\ p & 1 & 0 & 1 & - 1 & 0 & 0 & 0 & 0 & 0 & 0 & 0 & - 1 & 0 & 0 & 0 & 0 & 0 & 0 \\ aext & 0 & 0 & 0 & 0 & 0 & 0 & 1 & 0 & 0 & 0 & 0 & 0 & - 1 & 0 & 0 & 0 & 0 & 0 \\ bext & 0 & 0 & 0 & 0 & 0 & 0 & 0 & 1 & 0 & 0 & 0 & 0 & 0 & - 1 & 0 & 0 & 0 & 0 \\ cext & 0 & 0 & 0 & 0 & 0 & 0 & 0 & 0 & 1 & 0 & 0 & 0 & 0 & 0 & - 1 & 0 & 0 & 0 \\ dext & 0 & 0 & 0 & 0 & 0 & 0 & 0 & 0 & 0 & 1 & 0 & 0 & 0 & 0 & 0 & - 1 & 0 & 0 \\ gext & 0 & 0 & 0 & 0 & 0 & 0 & 0 & 0 & 0 & 0 & 1 & 0 & 0 & 0 & 0 & 0 & - 1 & 0 \\ pext & 0 & 0 & 0 & 0 & 0 & 0 & 0 & 0 & 0 & 0 & 0 & 1 & 0 & 0 & 0 & 0 & 0 & - 1\end{matrix}$$

The following lb- and ub-vectors of the toy model define the flux lower and upper bounds, corresponding to thermodynamic or capacity constraints on the respective flux vector components. Values of—1000 and 1000 represent, in this sample calculation, unlimited flux bounds:

$$\begin{matrix}lb = 0 \\ 0 \\ 0 \\ 0 \\ 0 \\ 0 \\ - 1000 \\ - 1000 \\ - 1000 \\ - 1000 \\ - 1000 \\ - 1000 \\ 0 \\ 0 \\ 0 \\ 0 \\ 0 \\ 0\end{matrix} \begin{matrix}ub = 1000 \\ 1000 \\ 1000 \\ 1000 \\ 1000 \\ 1000 \\ 1000 \\ 1000 \\ 1000 \\ 1000 \\ 1000 \\ 1000 \\ 0 \\ 0 \\ 0 \\ 0 \\ 1000 \\ 1000\end{matrix}$$

The following c-vector of the toy model encodes the biological objective of the model:

$$\begin{matrix} 0 \\ 0 \\ 0 \\ 0 \\ 0 \\ 0 \\ 0 \\ 0 \\ 0 \\ 0 \\ 0 \\ 0 \\ 0 \\ 0 \\ 0 \\ 0 \\ 1 \\ 0\end{matrix}$$

Obviously, with the only non-zero component of the above c-vector referring to the flux vector component v17 related to the exchange reaction Gext (see FIG. 2), the biological objective of the toy model considered here is growth creating the extracellular compound Gext.

The following b-vector of the toy model defines the metabolite mass balances, that is balance of the 14 metabolites considered in the toy model. The assumption here is a steady state, such that the components of the b-vector are set to zeros:

$$\begin{matrix} 0 \\ 0 \\ 0 \\ 0 \\ 0 \\ 0 \\ 0 \\ 0 \\ 0 \\ 0 \\ 0 \\ 0 \\ 0 \\ 0\end{matrix}$$

It should be noted that the lb-vector of the toy model introduced above defines a lower bound for each component of a possible flux vector of the toy model. Similarly, the ub-vector of the toy model defined above introduces for each component of a possible flux vector of the toy model an upper bound. In more elaborate scenarios than the toy model considered here, the values for the lower and upper bounds of the components of possible flux vectors may be determined experimentally or may be determined from thermodynamic constraints.

Regarding the c-vector above related to the biological objective, this objective may vary in different models. For instance, in a larger model, the biological objective may not necessarily be growth but for instance a maximization of ATP generation.

Finally, simulation of a steady state, incorporated with a b-vector with all components set to zero as indicated for the case of the toy model above, is one convenient way to simulate evolution of a cell or organism in a chemical environment, thereby assuming that the system is in a steady-state characterised by the above described mass balance.

A quantitative determination of the suitability of a chemical environment according to an embodiment of the invention is based on the knowledge of and the subsequent comparison with a reference situation. In context of the invention, a comparison to a reference scenario is intended in order to quantitatively rate the suitability or potential of a chemical environment. In particular, with respect to consideration of a worst-case coverage according to the invention, reference state flux ranges may be considered and compared to fluxes in the respective model. Accordingly, in the toy model calculation, reference state fluxes are needed in context of consideration of a worst-case coverage according to the invention.

The worst-case coverage and the calculation associated therewith is considered later on. Here, the reference state for the toy model calculation is defined to be a state with growth on the substrates A and B, compare FIG. 2.

The reference state is described by the minimum and maximum values of each of the fluxes as following:

| | min | max |
|---|---|---|
| v1 | 0 | 0 |
| v2 | 10 | 10 |
| v3 | 0 | 0 |
| v4 | 0 | 0 |
| v5 | 10 | 10 |
| v6 | 10 | 10 |
| v7 | −10 | −10 |
| v8 | −10 | −10 |
| v9 | 0 | 0 |
| v10 | 0 | 0 |
| v11 | 10 | 10 |
| v12 | 0 | 0 |
| v13 | −10 | −10 |
| v14 | −10 | −10 |
| v15 | 0 | 0 |
| v16 | 0 | 0 |
| v17 | 10 | 10 |
| v18 | 0 | 0 |

After this preparation, method steps according to embodiments of the invention are now explained with reference to the toy model. To this end, the suitability or potential of a chemical environment containing substrates C and D to up-regulate flux through reaction v3 (i.e. v3 is the up-regulation target) shall be assessed.

It should be noted that in order to simplify the situation, no inhibition is considered within the toy model.

In a first step, a growth optimality condition is set using linear optimization as follows:

min Σνᵤₚₜₐₖₑ, (1)

subject to the following constraints:

S·ν=0, (2)

ν_{μ}=10, (3)

ν_{inhibited}=0, (4)

ν_{lb}≤ν≤ν_{ub}. (5)

Here νᵤₚₜₐₖₑ, denote the fluxes of the uptake reactions of the components available in the particular chemical environment, S denotes the stoichiometric matrix of the genome-scale metabolic toy model of the species, ν denotes a flux vector, ν_{μ} is the growth rate (i.e. flux), ν_{inhibited} are the fluxes through the reactions inhibited by the inhibitors present in the particular chemical environment, ν_{lb} and ν_{ub} the flux lower and upper bounds, respectively.

It is noted that in most models the uptake fluxes are defined negative, such that in these situations the above minimization would be done on −νᵤₚₜₐₖₑ. The skilled person will easily adapt this situation without leaving the scope of the invention.

The above prescriptions given in equations (1) to (5) are implemented into the following form:

max f′x (I)

subject to the constraints

Aeq·x=beq, (II)

lbᵢ≤xᵢ≤ubᵢ, (III)

where the above introduced quantities Aeq, lb, ub, f and beq are given as follows: The matrix Aeq is given by the S-matrix of the toy model with two columns added, which refer to possible secretion of the components of the chemical environment.

This refers to the constraints in equation 2 above. Accordingly, Aeq is given by:

$$\begin{matrix}- 1 & - 1 & 0 & 0 & 0 & 0 & - 1 & 0 & 0 & 0 & 0 & 0 & 0 & 0 & 0 & 0 & 0 & 0 & 0 & 0 \\ 0 & 0 & 0 & 0 & - 1 & 0 & 0 & - 1 & 0 & 0 & 0 & 0 & 0 & 0 & 0 & 0 & 0 & 0 & 0 & 0 \\ 1 & 0 & - 1 & 0 & 0 & 0 & 0 & 0 & - 1 & 0 & 0 & 0 & 0 & 0 & 0 & 0 & 0 & 0 & 0 & 0 \\ 0 & 0 & 0 & - 1 & 0 & 0 & 0 & 0 & 0 & - 1 & 0 & 0 & 0 & 0 & 0 & 0 & 0 & 0 & 0 & 0 \\ 0 & 1 & 0 & 0 & - 1 & 0 & 0 & 0 & 0 & 0 & 0 & 0 & 0 & 0 & 0 & 0 & 0 & 0 & 0 & 0 \\ 0 & 0 & 0 & 1 & 1 & - 1 & 0 & 0 & 0 & 0 & 0 & 0 & 0 & 0 & 0 & 0 & 0 & 0 & 0 & 0 \\ 0 & 0 & 0 & 0 & 0 & 1 & 0 & 0 & 0 & 0 & - 1 & 0 & 0 & 0 & 0 & 0 & 0 & 0 & 0 & 0 \\ 1 & 0 & - 1 & 0 & 0 & 0 & 0 & 0 & - 1 & 0 & 0 & 0 & 0 & 0 & 0 & 0 & 0 & 0 & 0 & 0 \\ 0 & 0 & 1 & - 1 & 0 & 0 & 1 & 0 & 0 & 0 & 0 & - 1 & - 1 & 0 & 0 & 0 & 0 & 0 & 0 & 0 \\ 0 & 0 & 0 & 0 & 0 & 0 & 0 & 1 & 0 & 0 & 0 & 0 & 0 & - 1 & 0 & 0 & 0 & 0 & 0 & 0 \\ 0 & 0 & 0 & 0 & 0 & 0 & 0 & 0 & 1 & 0 & 0 & 0 & 0 & 0 & - 1 & 0 & 0 & 0 & - 1 & 0 \\ 0 & 0 & 0 & 0 & 0 & 0 & 0 & 0 & 0 & 1 & 0 & 0 & 0 & 0 & 0 & - 1 & 0 & 0 & 0 & - 1 \\ 0 & 0 & 0 & 0 & 0 & 0 & 0 & 0 & 0 & 0 & 1 & 0 & 0 & 0 & 0 & 0 & - 1 & 0 & 0 & 0 \\ 0 & 0 & 0 & 0 & 0 & 0 & 0 & 0 & 0 & 0 & 0 & 1 & 0 & 0 & 0 & 0 & 0 & - 1 & 0 & 0\end{matrix}$$

The vectors lb, ub and f introduced in equations (I) to (III) are given by:

$$\begin{matrix}lb = 0 \\ 0 \\ 0 \\ 0 \\ 0 \\ 0 \\ - 1000 \\ - 1000 \\ - 1000 \\ - 1000 \\ - 1000 \\ - 1000 \\ 0 \\ 0 \\ - 1000 \\ - 1000 \\ 10 \\ 0 \\ 0 \\ 0\end{matrix} \begin{matrix}ub = 1000 \\ 1000 \\ 1000 \\ 1000 \\ 1000 \\ 1000 \\ 1000 \\ 1000 \\ 1000 \\ 1000 \\ 1000 \\ 1000 \\ 0 \\ 0 \\ 0 \\ 0 \\ 10 \\ 1000 \\ 1000 \\ 1000\end{matrix} \begin{matrix}f = 0 \\ 0 \\ 0 \\ 0 \\ 0 \\ 0 \\ 0 \\ 0 \\ 0 \\ 0 \\ 0 \\ 0 \\ 0 \\ 0 \\ 1 \\ 1 \\ 0 \\ 0 \\ 0 \\ 0\end{matrix}$$

The last three rows of lb, ub and f represent the constraints for the possible secretion of the components of the chemical environment, which refers to the constraints in Equation 3.

The value l in vector f above represent the uptakes of the components of chemical environment in the objective, corresponding to constraints in Equation 1.

The 5th and the 6th last row of lb and up represent the free uptakes of the components of chemical environment allowed.

The following vector beq represents the b-vector of the toy model, corresponding to constraints in Equation 2:

$$\begin{matrix} 0 \\ 0 \\ 0 \\ 0 \\ 0 \\ 0 \\ 0 \\ 0 \\ 0 \\ 0 \\ 0 \\ 0 \\ 0 \\ 0\end{matrix}$$

The above system of equations is solved with, for instance, the CPLEX LP-solver or GLPK solver. The solver returns values of the variables corresponding to the optimal value of the objective function. It is worth to note that x may not be unique in real cases. However, the optimal value of objective function (i.e. f′x) is unique. In the toy model considered here, the following—in this case unique—values for the components of x are obtained:

$$\begin{matrix} 0 \\ 0 \\ 10 \\ 10 \\ 0 \\ 10 \\ 0 \\ 0 \\ - 10 \\ - 10 \\ 10 \\ 0 \\ 0 \\ 0 \\ - 10 \\ - 10 \\ 10 \\ 0 \\ 0 \\ 0\end{matrix}$$

In the next method step in the toy model calculation, the simulation of worst-case selection pressure on the target reactions under the growth-optimality constraint can be considered according to the equations:

min Σ|νᵤₚ|+Σ−|ν_{down}|, (6)

subject to

S·ν=0, (7)

ν_{μ}=10, (8)

ν_{inhibtted}=0, (9)

ν_{lb}≤ν≤ν_{ub}, (10)

Σνᵤₚₜₐₖₑ≤min Σνᵤₚₜₐₖₑ. (11)

Here, νᵤₚ and ν_{down} are fluxes through the up- and down-regulation target reactions, respectively.

In the particular example of the toy model considered here, the target flux is not reversible. Therefore, there is no need to add variables and constraints to implement the optimization of the absolute values of fluxes as a mixed-integer linear programming problem here. However, in case of more elaborate scenarios, this might be necessary.

The above constraints are brought into the following form:

max f′x (IV)

subject to

Aeq·x=beq, (V)

A·x≤b, (VI)

lbᵢ≤x≤ubᵢ. (VII)

The matrix Aeq in equation (IV) is given by the S-matrix of the model with two columns for flux variables added to describe the possible secretion of the components of the chemical environment. This corresponds to the constraints in equation 7 above. Accordingly, the matrix Aeq reads:

$$\begin{matrix}- 1 & - 1 & 0 & 0 & 0 & 0 & - 1 & 0 & 0 & 0 & 0 & 0 & 0 & 0 & 0 & 0 & 0 & 0 & 0 & 0 \\ 0 & 0 & 0 & 0 & - 1 & 0 & 0 & - 1 & 0 & 0 & 0 & 0 & 0 & 0 & 0 & 0 & 0 & 0 & 0 & 0 \\ 1 & 0 & - 1 & 0 & 0 & 0 & 0 & 0 & - 1 & 0 & 0 & 0 & 0 & 0 & 0 & 0 & 0 & 0 & 0 & 0 \\ 0 & 0 & 0 & - 1 & 0 & 0 & 0 & 0 & 0 & - 1 & 0 & 0 & 0 & 0 & 0 & 0 & 0 & 0 & 0 & 0 \\ 0 & 1 & 0 & 0 & - 1 & 0 & 0 & 0 & 0 & 0 & 0 & 0 & 0 & 0 & 0 & 0 & 0 & 0 & 0 & 0 \\ 0 & 0 & 0 & 1 & 1 & - 1 & 0 & 0 & 0 & 0 & 0 & 0 & 0 & 0 & 0 & 0 & 0 & 0 & 0 & 0 \\ 0 & 0 & 0 & 0 & 0 & 1 & 0 & 0 & 0 & 0 & - 1 & 0 & 0 & 0 & 0 & 0 & 0 & 0 & 0 & 0 \\ 1 & 0 & - 1 & 0 & 0 & 0 & 0 & 0 & - 1 & 0 & 0 & 0 & 0 & 0 & 0 & 0 & 0 & 0 & 0 & 0 \\ 0 & 0 & 1 & - 1 & 0 & 0 & 1 & 0 & 0 & 0 & 0 & - 1 & - 1 & 0 & 0 & 0 & 0 & 0 & 0 & 0 \\ 0 & 0 & 0 & 0 & 0 & 0 & 0 & 1 & 0 & 0 & 0 & 0 & 0 & - 1 & 0 & 0 & 0 & 0 & 0 & 0 \\ 0 & 0 & 0 & 0 & 0 & 0 & 0 & 0 & 1 & 0 & 0 & 0 & 0 & 0 & - 1 & 0 & 0 & 0 & - 1 & 0 \\ 0 & 0 & 0 & 0 & 0 & 0 & 0 & 0 & 0 & 1 & 0 & 0 & 0 & 0 & 0 & - 1 & 0 & 0 & 0 & - 1 \\ 0 & 0 & 0 & 0 & 0 & 0 & 0 & 0 & 0 & 0 & 1 & 0 & 0 & 0 & 0 & 0 & - 1 & 0 & 0 & 0 \\ 0 & 0 & 0 & 0 & 0 & 0 & 0 & 0 & 0 & 0 & 0 & 1 & 0 & 0 & 0 & 0 & 0 & - 1 & 0 & 0\end{matrix}$$

The constraints introduced by equation 8 and equation 11 referring to quantity A in equation (VI) can be written as

$$\begin{matrix}0 & 0 & 0 & 0 & 0 & 0 & 0 & 0 & 0 & 0 & 0 & 0 & 0 & 0 & 0 & 0 & - 1 & 0 & 0 & 0 \\ 0 & 0 & 0 & 0 & 0 & 0 & 0 & 0 & 0 & 0 & 0 & 0 & 0 & 0 & - 1 & - 1 & 0 & 0 & 0 & 0\end{matrix}$$

where the first line refers to the constraint from equation 8 and the second line to the constraint from equation 11.

Further, the following vectors lb, up and f arise in this method step within the toy model:

$$l b = \begin{matrix}0 \\ 0 \\ 0 \\ 0 \\ 0 \\ 0 \\ - 1000 \\ - 1000 \\ - 1000 \\ - 1000 \\ - 1000 \\ - 1000 \\ 0 \\ 0 \\ - 1000 \\ - 1000 \\ 10 \\ 0 \\ 0 \\ 0\end{matrix} ub = \begin{matrix}1000 \\ 1000 \\ 1000 \\ 1000 \\ 1000 \\ 1000 \\ 1000 \\ 1000 \\ 1000 \\ 1000 \\ 1000 \\ 1000 \\ 0 \\ 0 \\ 0 \\ 0 \\ 10 \\ 1000 \\ 1000 \\ 1000\end{matrix} f = \begin{matrix}0 \\ 0 \\ 1 \\ 0 \\ 0 \\ 0 \\ 0 \\ 0 \\ 0 \\ 0 \\ 0 \\ 0 \\ 0 \\ 0 \\ 0 \\ 0 \\ 0 \\ 0 \\ 0 \\ 0\end{matrix}$$

From the constraint in equation 7 the following b-vector beq results:

$$\begin{matrix}0 \\ 0 \\ 0 \\ 0 \\ 0 \\ 0 \\ 0 \\ 0 \\ 0 \\ 0 \\ 0 \\ 0 \\ 0 \\ 0\end{matrix}$$

Finally, the constrains from equations 8 and 11 translated in the form of equation (VI) give rise to the following value of b in equation (VI):

$$\begin{matrix}- 10 \\ 20\end{matrix}$$

Again, the problem is solved with, for instance, either a CPLEX LP or CPLEX MILP solver depending on whether there were reversible fluxes (i.e. fluxes with a lower bound smaller than 0, lb<0) involved in the targets. In the toy model case the solver returns as x (i.e. values of the variables) corresponding to the optimal value of the objective function the following vector x:

$$\begin{matrix}0 \\ 0 \\ 10 \\ 10 \\ 0 \\ 10 \\ 0 \\ 0 \\ - 10 \\ - 10 \\ 10 \\ 0 \\ 0 \\ 0 \\ - 10 \\ - 10 \\ 10 \\ 0 \\ 0 \\ 0\end{matrix}$$

It should again be noted that x may not be unique in real cases, only the optimal value of objective function (i.e. f′x) is unique.

According to the above result, the optimal value of the objective function (i.e. f′x), which is related to the score of the strength of the selection pressure created by the particular chemical environment, is given by 10 in the toy model case considered here.

Next, the simulation of a worst-case target reaction coverage under the growth-optimality and worst-case selection pressure constraints can be performed within the toy model as follows:

min Σb_{targets} (12)

subject to

s˜ν=0, (13)

ν_{μ}=10, (14)

ν_{inhibited}=0, (15)

ν_{lb}≤ν≤ν_{ub} (16)

Σνᵤₚₜₐₖₑ≤min≤Σνᵤₚₜₐₖₑ, (17)

|νᵤₚ|+Σ−|σ_{down}|≤min(Σ|νᵤₚ|+E−|ν_{down}), (18)

|νᵤₚ|−((1+δ)·w_{ub}−|νᵤₚ|_{ub})·(1−b_{targets})≤|νᵤₚ|_{ub} (19)

|ν_{down}−((1−δ)·w_{lb}−|ν_{down}|_{lb})·(1−b_{targets})≥|ν_{down}|_{lb} (20)

b_{targets}ϵ{0,1}. (21)

Here, b_{targets} are binary variables defining whether the absolute fluxes through the target reactions are beyond a corresponding thresholds defined from absolute flux ranges under reference conditions (i.e. w_{lb}, w_{ub}) or not. The case of the reference scenario with its reference conditions for the toy model calculation has been considered above and the values obtained for this scenario will now be used in the toy model calculation.

The parameter δ in equations (19) and (20) above is a threshold parameter stating how much lower or higher a flux relative to growth should be in the particular chemical environment in order to consider the corresponding target reaction to become exposed to (i.e. covered by) the selection pressure. Accordingly, by equations (19) and (20), an example for quantitative determination of a coverage of the target reaction in the toy scenario is introduced. Together with the constraints from equation (18), a worst-case coverage scenario is therefore considered by the set of equations (12) to (21).

For explicit calculation, the above constraints are brought into the following form, where x may contain both continuous and integer variables:

max f′x (VIII)

subject to

Aeq−x=beq, (IX)

A·x≤b, (X)

lbᵢ≤xᵢ≤ubᵢ, (XI)

x=[x_{b},x_{c}], (XII)

x_{b}∈{0,1}. (XIII)

The quantities in the equations (VIII) to (XIII) above are given by the following expressions.

Aeq represents the S-matrix of the model again with two columns added to describe the possible secretion of the components of the chemical environment, and with an additional column added for a coverage variable of the target reaction, reflecting the constraints in equation 13. Accordingly, Aeq in equation (IX) reads:

$$\begin{matrix}- 1 & - 1 & 0 & 0 & 0 & 0 & - 1 & 0 & 0 & 0 & 0 & 0 & 0 & 0 & 0 & 0 & 0 & 0 & 0 & 0 & 0 \\ 0 & 0 & 0 & 0 & - 1 & 0 & 0 & - 1 & 0 & 0 & 0 & 0 & 0 & 0 & 0 & 0 & 0 & 0 & 0 & 0 & 0 \\ 1 & 0 & - 1 & 0 & 0 & 0 & 0 & 0 & - 1 & 0 & 0 & 0 & 0 & 0 & 0 & 0 & 0 & 0 & 0 & 0 & 0 \\ 0 & 0 & 0 & - 1 & 0 & 0 & 0 & 0 & 0 & - 1 & 0 & 0 & 0 & 0 & 0 & 0 & 0 & 0 & 0 & 0 & 0 \\ 0 & 1 & 0 & 0 & - 1 & 0 & 0 & 0 & 0 & 0 & 0 & 0 & 0 & 0 & 0 & 0 & 0 & 0 & 0 & 0 & 0 \\ 0 & 0 & 0 & 1 & 1 & - 1 & 0 & 0 & 0 & 0 & 0 & 0 & 0 & 0 & 0 & 0 & 0 & 0 & 0 & 0 & 0 \\ 0 & 0 & 0 & 0 & 0 & 1 & 0 & 0 & 0 & 0 & - 1 & 0 & 0 & 0 & 0 & 0 & 0 & 0 & 0 & 0 & 0 \\ 1 & 0 & - 1 & 0 & 0 & 0 & 0 & 0 & - 1 & 0 & 0 & 0 & 0 & 0 & 0 & 0 & 0 & 0 & 0 & 0 & 0 \\ 0 & 0 & 1 & - 1 & 0 & 0 & 1 & 0 & 0 & 0 & 0 & - 1 & - 1 & 0 & 0 & 0 & 0 & 0 & 0 & 0 & 0 \\ 0 & 0 & 0 & 0 & 0 & 0 & 0 & 1 & 0 & 0 & 0 & 0 & 0 & - 1 & 0 & 0 & 0 & 0 & 0 & 0 & 0 \\ 0 & 0 & 0 & 0 & 0 & 0 & 0 & 0 & 1 & 0 & 0 & 0 & 0 & 0 & - 1 & 0 & 0 & 0 & - 1 & 0 & 0 \\ 0 & 0 & 0 & 0 & 0 & 0 & 0 & 0 & 0 & 1 & 0 & 0 & 0 & 0 & 0 & - 1 & 0 & 0 & 0 & - 1 & 0 \\ 0 & 0 & 0 & 0 & 0 & 0 & 0 & 0 & 0 & 0 & 1 & 0 & 0 & 0 & 0 & 0 & - 1 & 0 & 0 & 0 & 0 \\ 0 & 0 & 0 & 0 & 0 & 0 & 0 & 0 & 0 & 0 & 0 & 1 & 0 & 0 & 0 & 0 & 0 & - 1 & 0 & 0 & 0\end{matrix}$$

The quantity A in equation (X) above encodes the constraints in equations 14, 17 and 19, and is therefore given by:

$$\begin{matrix}0 & 0 & 0 & 0 & 0 & 0 & 0 & 0 & 0 & 0 & 0 & 0 & 0 & 0 & 0 & 0 & - 1 & 0 & 0 & 0 & 0 \\ 0 & 0 & 0 & 0 & 0 & 0 & 0 & 0 & 0 & 0 & 0 & 0 & 0 & 0 & - 1 & - 1 & 0 & 0 & 0 & 0 & 0 \\ 0 & 0 & 1 & 0 & 0 & 0 & 0 & 0 & 0 & 0 & 0 & 0 & 0 & 0 & 0 & 0 & 0 & 0 & 0 & 0 & - 1000 \\ 0 & 0 & 1 & 0 & 0 & 0 & 0 & 0 & 0 & 0 & 0 & 0 & 0 & 0 & 0 & 0 & 0 & 0 & 0 & 0 & 0\end{matrix}$$

The vectors lb, ub, and f are given by the following expressions:

$$l b = \begin{matrix}0 \\ 0 \\ 0 \\ 0 \\ 0 \\ 0 \\ - 1000 \\ - 1000 \\ - 1000 \\ - 1000 \\ - 1000 \\ - 1000 \\ 0 \\ 0 \\ - 1000 \\ - 1000 \\ 10 \\ 0 \\ 0 \\ 0 \\ 0\end{matrix} ub = \begin{matrix}1000 \\ 1000 \\ 1000 \\ 1000 \\ 1000 \\ 1000 \\ 1000 \\ 1000 \\ 1000 \\ 1000 \\ 1000 \\ 1000 \\ 0 \\ 0 \\ 0 \\ 0 \\ 10 \\ 1000 \\ 1000 \\ 1000 \\ 1\end{matrix} f = \begin{matrix}0 \\ 0 \\ 0 \\ 0 \\ 0 \\ 0 \\ 0 \\ 0 \\ 0 \\ 0 \\ 0 \\ 0 \\ 0 \\ 0 \\ 0 \\ 0 \\ 0 \\ 0 \\ 0 \\ 0 \\ 1\end{matrix} .$$

Further, the following variable types (C=continuous; B=binary) for the components of flux vectors in this calculation step are defined to be:

$$\begin{matrix}C \\ C \\ C \\ C \\ C \\ C \\ C \\ C \\ C \\ C \\ C \\ C \\ C \\ C \\ C \\ C \\ C \\ C \\ C \\ C \\ B\end{matrix}$$

The constraints from equation 13 are represented by the following vector beq:

$$\begin{matrix}0 \\ 0 \\ 0 \\ 0 \\ 0 \\ 0 \\ 0 \\ 0 \\ 0 \\ 0 \\ 0 \\ 0 \\ 0 \\ 0\end{matrix}$$

Finally, the quantity b in equation (X), representing constraints in equations 14, 17 and 19 is given by:

$$\begin{matrix}- 10 \\ 20 \\ 0 \\ 10\end{matrix}$$

The problem as given by the equations (VIII) to (XIII) is then solved with e.g. the CPLEX MILP solver.

In case of the toy model the solver returns as x (i.e. values of the variables) corresponding to the optimal value of the objective function the following vector:

$$\begin{matrix}0 \\ 0 \\ 10 \\ 10 \\ 0 \\ 10 \\ 2.27 E - 13 \\ 0 \\ - 10 \\ - 10 \\ 10 \\ 2.27 E - 13 \\ 0 \\ 0 \\ 1\end{matrix}$$

Thus, the optimal value of the objective function (i.e. f′x), which corresponds to the score of the coverage of the targets by the selection pressure created by the particular chemical environment, is 1 (the value in the last component of the above vector). This value implies that the only target in this case is covered even in worst-case.

Accordingly, in the toy model case, the chemical environment containing C and D is expected to create stronger selection pressure on the increase of the target flux than the reference chemical environment.

A combined numerical score can be derived from the worst-case selection pressure, the worst-case coverage, and the number of components in the particular chemical environment:

$$value = {W}_{c} \cdot \frac{( n - coverage )}{n} + {W}_{s} \cdot \frac{( 1000 \cdot u - strength )}{n} + {W}_{m} \cdot c \qquad ( 22 )$$

value is the combined score (i.e. the lower the value the better the particular chemical environment is to evolve the trait), n is the number of target reactions, coverage is the worst-case coverage (i.e. min Σb_{targets}), u is the number of up-regulation target reactions, strength is the worst-case selection pressure strength (i.e. min Σ|νᵤₚ|+Σ−|ν_{down}|), c is the number of components in the particular chemical environment, and W_{c}, Wₛ, Wₘ are weights to be assigned for the coverage, strength, and the complexity of the chemical environment, respectively.

In the toy model case, setting the weight to W_{c}=1000, Wₛ=1, and Wₘ=1, the combined score of the chemical environment containing compounds C and D is obtained to be 992.

If several chemical environments were evaluated, this score would then be compared against the scores of the other chemical environments to choose a suitable chemical environment out of the set of considered chemical environments. Generally, the lower the score the better suited is the particular chemical environment to evolve a particular desired metabolic trait. The skilled person will easily adapt the scoring formula, for example so that the higher the score the better the suitability, without leaving the scope of the invention.

The design of the chemical environment could also be carried out by performing optimization of the combination of components from a set of possible components e.g. using the method according to the invention as a fitness function in a genetic algorithm.

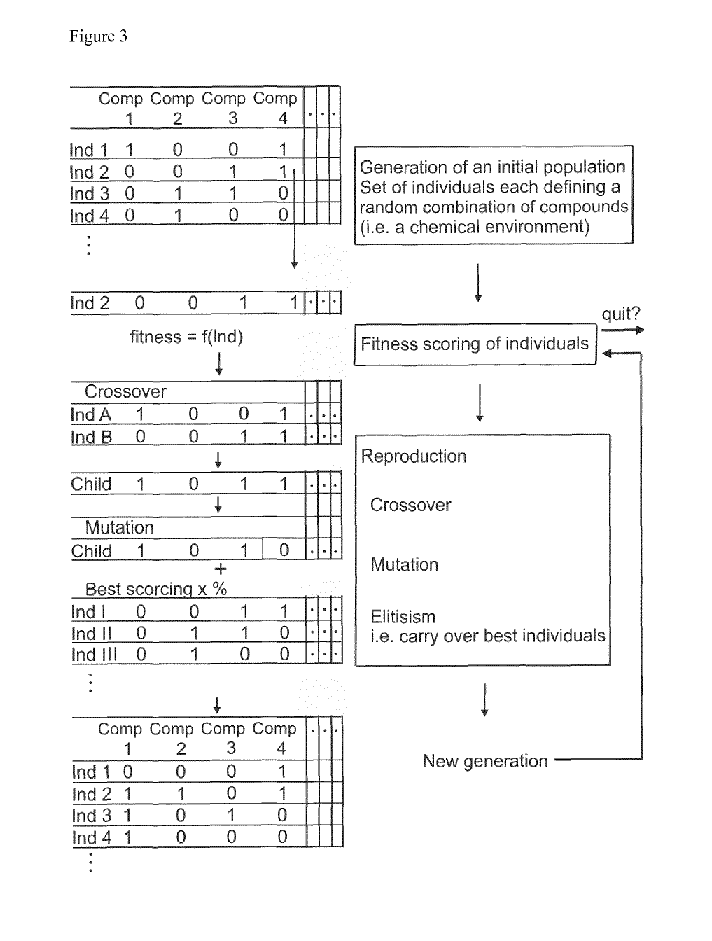

With respect to FIG. 3 an example for an application of a method according to the invention in context of a genetic algorithm is given. The algorithm is suited for the design of optimal chemical environments for an adaptive evolution of a metabolic trait. Individuals of the population in the genetic algorithm are particular chemical environments, defined as binary vectors representing the combinations of compounds. The fitness scoring, evaluates the goodness of an individual chemical environment for adaptive evolution of the metabolic trait. Reproduction phase creates a new generation of individuals through crossovers, mutations, and elitism.

### EvolveX Case Study 1. Improved Generation of Wine Aromas

The case study aimed to evolve Saccharomyces cerevisiae to gain two desired metabolic traits in a target niche of wine must: 1) an increased generation of Phenylethyl acetate (i.e. an ester of Phenylethyl alcohol and Acetate having a rose and honey scent and raspberry-like flavour) and 2) an increased generation of aromas derived from branched-chain amino acids or their precursors. These traits were further defined in terms of up- and down-regulation target fluxes with respect to the initial phenotype. They were identified using a genome-scale metabolic model of S. cerevisiae as fluxes that in minimum (i.e. included in the minimum number of fluxes) are necessary to change beyond a threshold to obtain the desired trait. As an initial metabolic phenotype, S. cerevisiae flux distribution when growing on excess glucose with ammonium as a nitrogen source (instead of a complex mixture of nitrogen sources in wine must) was used to identify also the targets enhancing the de novo generation of the amino acid derived aromas. To enable the adaptive evolution of these desired traits (i.e. the specific up- and down-regulations of target fluxes) with natural selection, Evolution niche needed to be designed such that when the cells grow in Evolution niche the desired trait is growth-enhancing (i.e. fitness-beneficial). Thus, when exposed to the Evolution niche for prolonged time the population of cells will enrich in mutations that enable or strengthen the desired traits in the Target niche.

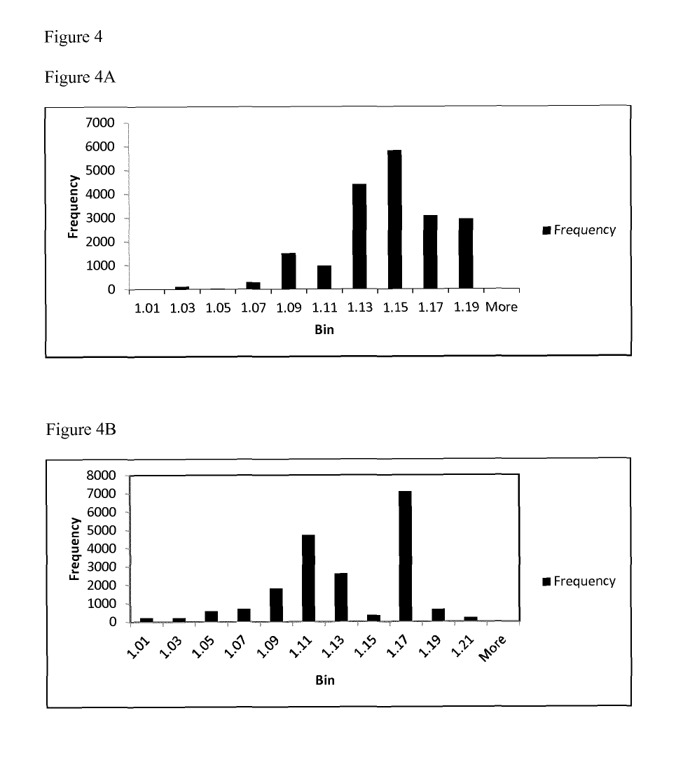

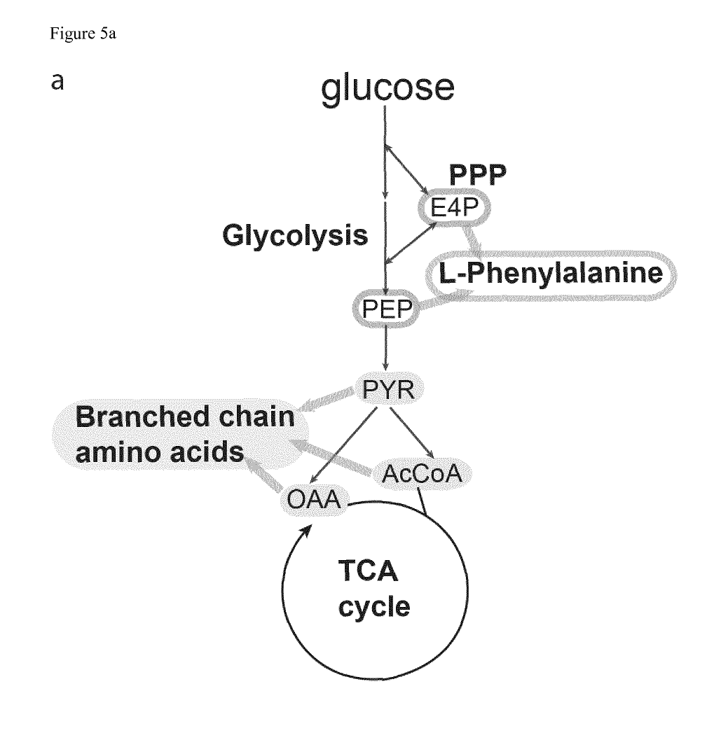

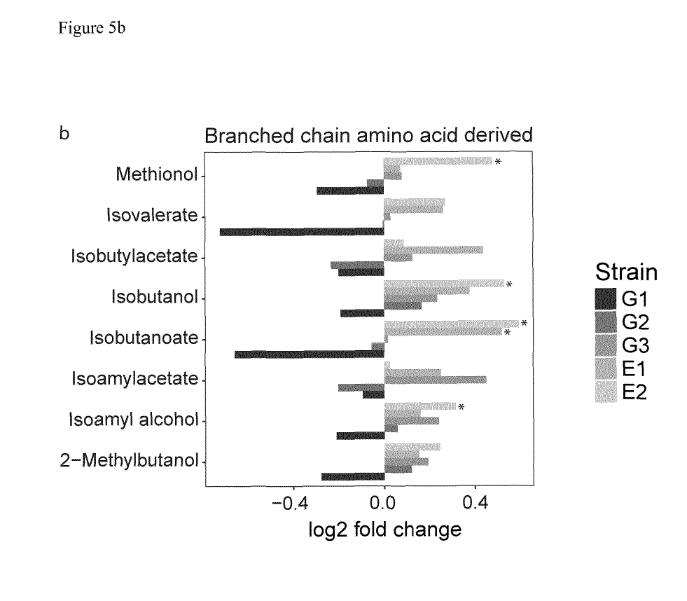

EvolveX algorithm was then used to evaluate the suitability of particular chemical environments as an Evolution niche for an adaptive evolution of the desired traits for a Target niche. The genome-scale metabolic model of S. cerevisiae was used to predict the relative strength of selection pressure on target reaction fluxes under a given Evolution niche defined by its chemical composition. EvolveX can consider niches composed of number of different molecules including carbon and nitrogen sources that can be utilized up by the organism (i.e. uptake and utilization described in the model) and inhibitors (e.g. substrate analogs) or regulatory triggers (e.g. 2-deoxyglucose) whose effect on specific metabolic reactions can be modelled. If a reference niche is defined (e.g. common growth conditions), EvolveX calculates also the coverage of target reactions by selection pressure stronger than under the reference conditions. Further, EvolveX derives a combined score for a particular Evolution niche from the selection pressure on the target fluxes, their coverage by selection pressure stronger than under reference conditions, and the number of chemical components in the niche. Here, EvolveX was run to score the chemical environments formed as all the combinations of three of the common carbon and nitrogen sources defined in the genome-scale metabolic model of S. cerevisiae and additionally including gluconate. As the reference conditions, glucose minimal laboratory medium was used. The well-scoring (i.e. the lower the better for the combined score) environments were manually inspected to choose Evolution niches for experimental adaptive evolutions. For evolving an increased generation of Phenylethyl acetate the following medium was chosen: Glycerol, L-Phenylalanine, L-Threonine, and for evolving an increased generation of aromas derived from branched chain amino acids the following medium was chosen: Ethanol, Glycine L-Arginine. These combinations of compounds served as the sole carbon and nitrogen sources in the adaptive evolution experiments. Ethanol, Glycine, L-Arginine combination chosen scored well (1.04 relative to the minimum score observed) for the suitability for evolving an increased generation of branched chain aromatic acids derived esters as can be seen in the histogram (FIG. 4A) and was chosen over the lower scoring ones because Ethanol as a carbon source can support fast growth and thus would facilitate the experiments. Glycerol, L-Phenylalanine, and L-Threonine scored relatively well (1.06 relative to the minimum score observed) for the suitability for evolving an increased generation of Phenylethyl acetate (FIG. 4B). This combination was chosen over the better scoring ones because the initial experiments showed that D-Ribose and Gluconate would not sustain growth as carbon sources and a sole contribution of amino acids as carbon and nitrogen sources was also expected not to sustain growth.

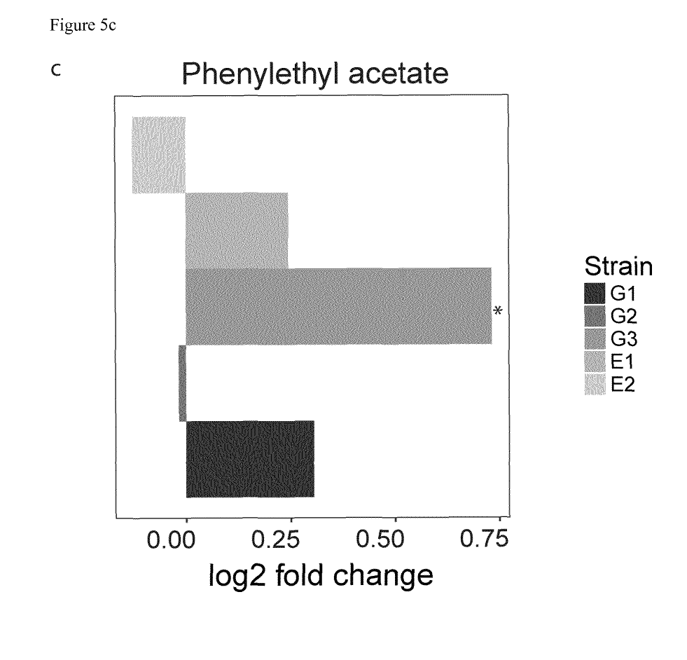

The experimental adaptive evolution was performed in small scale liquid cultures. After serial transfers in the chosen Evolution niches in three replicate lineages for approximately 80 generations, 9 single strains were isolated from the final populations of the each lineage. Based on their performance with respect to the performance of the final population on the Evolution niche-medium, selected strains (one strain from each of the lineages evolved for the increased generation of Phenylethyl acetate, and one strain from two of the lineages evolved for the increased generation of branched chain amino acid derived aromas) were characterized in the Target niche i.e. in wine fermentation mimicking cultivations on wine must in triplicate. Samples were withdrawn for volatile aroma profiling using GC-MS. The target compound generation was found increased in strains from populations evolved in the corresponding EvolveX designed media. FIG. 3 shows the target compound generation in the evolved isolated strains relative to the parental strain: b) the branched chain amino acid derived aromas, c) Phenylethyl acetate. FIG. 5 further shows a) the origin and precursors of the target aroma compounds in the metabolism of S. cerevisiae and d) the Ehrlich pathway that generates the aromatic fusel alcohols and acids that can further react to generate esters.

EvolveX Case Study 2: EvolveX Designed Adaptive Evolution of S. cerevisiae to Enhance the Support of Lactic Acid Bacteria (LAB) Growth.

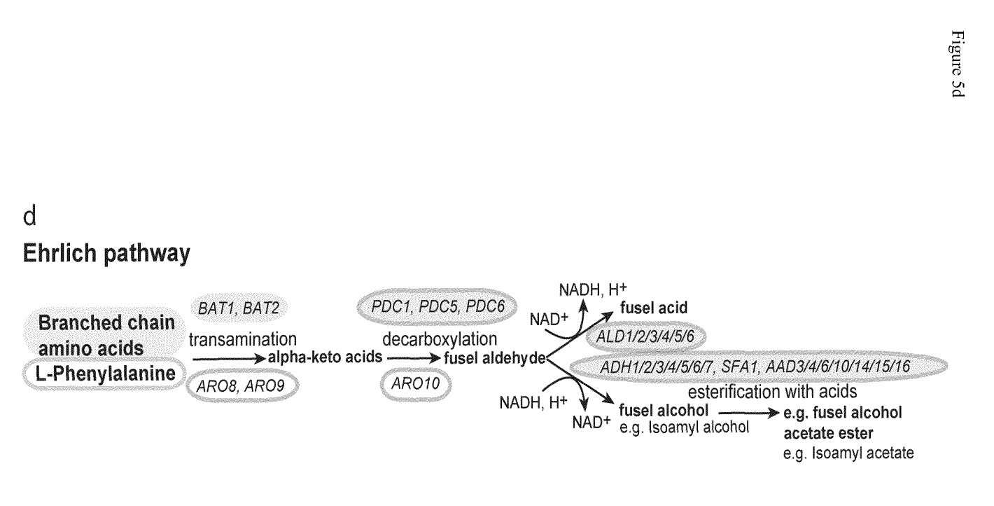

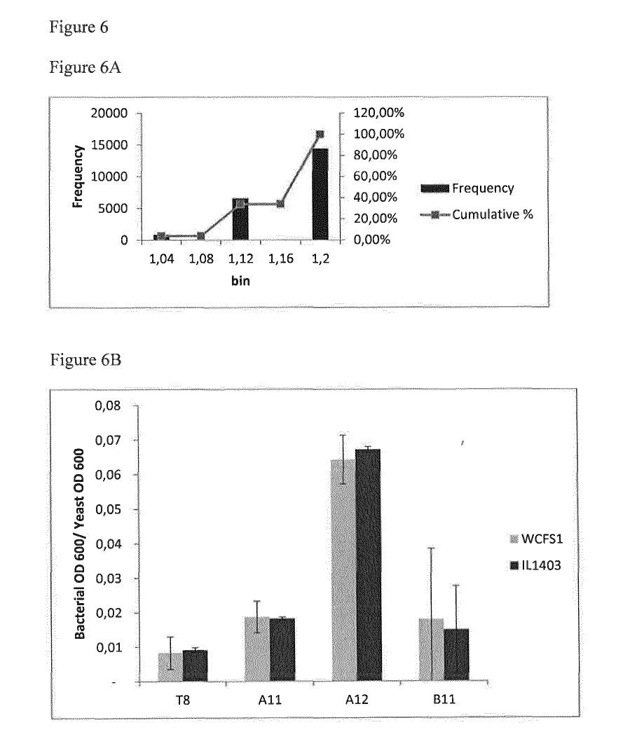

The second case study aimed to adaptively evolve S. cerevisiae for an altered exometabolome (i.e. secreted metabolites) to affect the growth support of LAB. We have previously observed that S. cerevisiae enables the growth of LAB on a medium that lacks essential amino acids. It was found that in a medium rich in nitrogen but imbalanced in the nature of N-sources, S. cerevisiae secretes nitrogen containing compounds, amino acids in particular. Here we aimed to use EvolveX to design conditions under which S. cerevisiae would become adaptively evolved to secrete an altered exometabolome to enhance the LAB growth. A genome-scale metabolic model of S. cerevisiae was used with additional constraints to reflect the regulation of amino acid biosynthesis. Specifically, 1) L-Tyrosine synthesis was constrained to be active even if L-Tyrosine was provided in the medium but not all the aromatic amino acids were available, 2) homoserine (L-Threonine precursor) synthesis was constrained to be active even in presence of L-Isoleucine or L-Threonine in the medium. In addition, free uptake of L-Isoleucine, L-Leucine, L-Valine and L-Tyrosine was allowed in the model. The target fluxes for EvolveX were set as follows: UP-regulation: L-threonine aldolase, pyruvate carboxylase, 4-hydroxyphenylpyruvate decarboxylase, 4-hydroxyphenylacetate dehydrogenase, tyrosol dehydrogenase, tyrosol secretion, and 4-hydroxyphenylacetate secretion; and for DOWN-regulation: pyruvate decarboxylase, and 4-hydroxypyruvate secretion. EvolveX algorithm was then used to score the chemical environments formed as all the combinations of three of the common carbon and nitrogen sources defined in the genome-scale metabolic model of S. cerevisiae and additionally including gluconate. As a reference chemical environment a glucose minimal medium with ammonium sulfate as N-source was used. The well-scoring (i.e. the lower the better for the combined score) environments were manually inspected to choose Evolution niches for experimental adaptive evolutions. From these, we selected a combination of 1) pyruvate and 2) L-Threonine (scoring 1.01 relative to the minimum score observed, for a score histogram of Evolution niches, see FIG. 6A).

Adaptive laboratory evolution was performed in growth plates on 1) pyruvate, 2) L-Threonine, 3) L-Isoleucine, 4) L-Leucine, 5) L-Valine, and 6) L-Tyrosine as sole C- and N-sources in buffered YNB (w/o N-source, NaOH+Succinic acid, pH 5.8) for around 100 generations during which the transfers by pinning to fresh plates were made every third day. From populations showing substantial growth improvement in the Evolution niche, single fast growing strains were isolated. The isolated strains were tested for their growth support to LABs (Lactococcus lactis, Lactobacillus plantarum) relative to the parental strain. A conditioned medium to test the growth support to LAB was prepared by growing yeast strains on CDM35 glucose medium (containing ammonium and amino acids: L-Arginine, L-Asparagine, L-Histidine, L-Methionine, L-Leucine, L-Isoleucine, L-Tyrosine, L-Valine; Ponomarova et al. manuscript under preparation), that as such does not enable the growth of the LABs. After 16 h or 20 h (i.e. from initial OD600 of 0.01) of incubation at 30° C. (w/o shaking) yeast cells were pelleted and discarded. The supernatants were further passed through a 0.22 mun sterile PVDF filter. Then, LABs were inoculated at initial OD600 of 0.01 in a 96-well plate into the yeast conditioned and non-conditioned media. LAB were incubated without shaking at 30° C. and their growth was assessed after four days. The three evolved strains showed an improved growth-support towards L. lactis (IL1403) and L. plantarum (WCSFI) (FIG. 6B).

The application also comprises the following embodiments:

### Embodiment 1

A method for evaluating the suitability of a chemical environment to evolve a desired metabolic trait of a cell or organism, the method comprising simulation of one or several of functions of fluxes in a metabolic model,
wherein the metabolic model comprises a stoichiometric representation of biochemical reactions and import and export of extracellular compounds,
   wherein the metabolic trait comprises a set of targets,
   wherein the targets are functions of fluxes.

### Embodiment 2

A method according to embodiment 1, wherein simulation of one or several targets relative to growth is performed,
wherein the growth is a function of fluxes through a reaction or reactions that generate biomass components or biomass.

### Embodiment 3

A method according to embodiment 2,
wherein a simulation is performed while the growth is constrained to a fixed value or within a range.

### Embodiment 4

A method according to embodiment 3,
wherein a simulation is performed while the growth is constrained to its optimal value.

### Embodiment 5

A method according to embodiment 3 or 4, wherein simulation is performed while a constraint is set on any of uptakes,
wherein uptakes are functions of fluxes through the reactions representing the import of extracellular compounds.

### Embodiment 6

A method according to embodiment 5, wherein a simulation is performed while a sum of uptakes is constrained to a fixed value or within a range.

### Embodiment 7

A method according to embodiment 6,
wherein a simulation is performed while a sum of uptakes is constrained to an optimal value.

### Embodiment 8

A method according to embodiment 7,
wherein a simulation is performed while any of uptakes is constrained to a fixed value or within a range.

### Embodiment 9

A method according to embodiment 8,
wherein a simulation is performed while any of uptakes is constrained to an optimal value.

### Embodiment 10

A method according to any of the preceding embodiments, wherein up-regulation targets and down-regulation targets are optimized into opposite directions,
wherein the up-regulation targets are those targets for which an increase is desired, and
wherein the down-regulation targets are those targets for which a decrease is desired.

### Embodiment 11

A method according to any of the preceding embodiments,
wherein simulation of absolute flux values is performed.

### Embodiment 12

A method according to any of the preceding embodiments,
wherein absolute values of up-regulation targets are minimized and absolute values of down-regulation targets are maximized.

### Embodiment 13

A method according to any of the preceding embodiments,
wherein the number of targets exceeding or falling below at least one threshold is optimized.

### Embodiment 14

A method according to any of the preceding embodiments,
wherein the number of targets relative to a growth exceeding or falling below at least one threshold is simulated.

### Embodiment 15

A method according to embodiment 13 or 14,
wherein the at least one threshold is determined with respect to functions of fluxes in a reference chemical environment.

### Embodiment 16

A method according to any of the preceding embodiments,
wherein in the simulation at least one inhibited flux is defined.

### Embodiment 17

A method according to any of the preceding embodiments,
wherein simulation is performed by constraining fluxes through reactions that are targets of inhibitors or regulatory triggers included in the chemical environment.

### Embodiment 18

A method according to any of the preceding embodiments,
wherein simulation is performed by constraining to zero fluxes through reactions that are targets of inhibitors or regulatory triggers included in the chemical environment.

### Embodiment 19

A method according to any of the preceding embodiments,
wherein a stoichiometric matrix of the metabolic model is used.

### Embodiment 20

A method according to any of the preceding embodiments, further comprising: determination of a numerical score for the chemical environment,
wherein the score is indicative for a strength of selection pressure on the targets in the chemical environment, and/or
   wherein the score is indicative for a coverage of the targets by the selection pressure in the chemical environment.

### Embodiment 21

A method according to any of the preceding embodiments, further comprising: determination of a numerical score for the chemical environment,
wherein the score is indicative for a strength of a worst-case selection pressure on the targets in the chemical environment,
   wherein the score is indicative for a worst-case coverage of the targets by the selection pressure in the chemical environment.

### Embodiment 22

A method according to embodiment 20 or 21,
wherein the score takes into account a number of components in the chemical environment in determining the score of the chemical environment.

### Embodiment 23

A method according to embodiment 22,
wherein the score comprises a function of a strength of the selection pressure and a coverage of the targets by the selection pressure, and a number of components in the chemical environment.

### Embodiment 24

A method according to any of the preceding embodiments, wherein simulation of one or several functions of fluxes through the target reactions relative to a growth is performed,

the method comprising the following steps:
(a) Performing a first optimization of the model imposing a plurality of constraints, thereby determining a first optimization result,
wherein a constraint sets a condition defining a growth to a fixed value or in range;
wherein a constraint sets thermodynamic bounds on fluxes in the model;
wherein the first optimization of the model optimizes a sum of uptakes,
(b) Performing a second optimization using the first optimization result, thereby determining a second optimization result,
   wherein a constraint sets a growth to a fixed value or in range;
   wherein a constraints sets a sum of uptakes to the first optimization result,
   wherein the second optimization result is indicative for the suitability of the chemical environment,
   wherein the second optimization of the model promotes a condition identified as optimizing a sum of up-regulation targets, and
   wherein the second optimization of the model promotes a condition identified as optimizing a sum of down-regulation targets.

### Embodiment 25

A method according to the preceding embodiments, further comprising the step:
(c) Performing a third optimisation of the model, thereby determining a third optimization result,
wherein the number of up-regulation targets which are enhanced with respect to up-regulation targets in a reference chemical environment are optimized, and
wherein the number of down-regulation targets which are suppressed with respect to down-regulation targets in a reference chemical environment are optimized.

### Embodiment 26

A method according to embodiments 24 and 25,
wherein the score is given by the value

$$value = {W}_{c} \cdot \frac{( n - coverage )}{n} + {W}_{s} \cdot \frac{( 1000 \cdot u - strength )}{n} + {W}_{m} \cdot c ,$$

wherein
n denotes the number of targets,
coverage denotes the minimal value of targets obtained in step (c),
u denotes the number of up-regulation targets,
strength denotes the sum of the minimised sum of up-regulation targets and the maximised sum of down-regulation targets obtained in step (b), and
c denotes the number of components in the chemical environment,
W_{c}, Wₛ, and Wₘ denote mathematical weights.

### Embodiment 27

A method according to any one of the preceding embodiments,
wherein the method is used for fitness scoring in a genetic algorithm.

### Embodiment 28

A method according to embodiment 27,
wherein the chemical environment represents an individual in the genetic algorithm, wherein the compounds of the chemical environment represent properties or genes of an individual in the genetic algorithm.

### Embodiment 29

A method according to any one of the preceding embodiments,
wherein the chemical environment is a natural environment for a cell or organism.

### Embodiment 30

A method for choosing a site to isolate a strain according to embodiment 29,
wherein the method leads to an isolation of a cell or organism with a metabolic trait.

### Embodiment 31

A method according to any one of the preceding embodiments,
wherein the metabolic trait leads to the production of at least one desired product by the cell or organism.

### Embodiment 32

A method according to embodiment 31,
wherein the at least one desired product is a food compound, preferably an aroma compound.

### Embodiment 33

A method according to embodiment 32,
wherein the at least one desired product is a polymer, an acid, an alcohol or an ester.

### Embodiment 34

A method according to any one of the preceding embodiments,
wherein the cell or organism is of industrial relevance.

### Embodiment 35

A method according to any one of the preceding embodiments,
wherein the cell or organism is a microorganism selected from the group of bacteria, yeast or molds.

### Embodiment 36

A method according to embodiment 35,
wherein the yeast is Saccharomyces cerevisiae.

### Embodiment 37

A method according to any one of the preceding embodiments,
wherein the chemical environment is a culture medium for the cell or organism.

### Embodiment 38

A method according to embodiments 1 to 37,
wherein the chemical environment is an evolution chemical environment used to evolve the metabolic trait of the cell or organism and differs from a target chemical environment used for culturing the cell or organism to produce at least one desired product.

### Embodiment 39

A method according to embodiments 1 to 37,
wherein the cell is a tumour cell.

### Embodiment 40

A method according to embodiments 1 to 37,
wherein the organism is a pathogen.

### Embodiment 41

A method according to embodiments 39 or 40,
wherein the metabolic trait leads to the destabilization of the cell or organism.

### Embodiment 42

A method according to embodiment 41,
wherein the destabilization of the cell or organism causes decreased growth and/or death of the cell or organism.

### Embodiment 43

A method for determining a chemical environment to evolve a metabolic trait of a cell or organism comprising the following steps:
(a) Evaluating the suitability to evolve a metabolic trait of at least two chemical environments using the method to any one of the preceding embodiments,
(b) Selecting at least one chemical environment with a suitability to evolve a metabolic trait, which belongs to the chemical environments with the highest potential to evolve a metabolic trait.

### Embodiment 44

Method according to embodiment 43, wherein in step (b) a chemical environment is selected which belongs to the 90%, 80%, 70%, 60%, 50%, 40% 30% 20%, 15%, 10%, 5%, 3%, 2% of chemical environments with the highest potential to evolve a metabolic trait.

### Embodiment 45

A method for determining a chemical environment to evolve a metabolic trait of a cell or organism comprising the following steps:
(a) Evaluating the suitability of at least two chemical environments using the method to any one of the preceding embodiments,
(b) Selecting at least one chemical environment with a suitability to evolve a metabolic trait, which exceeds a pre-set threshold of suitability.

### Embodiment 46

A method for evolving a metabolic trait of the cell or organism comprising the steps of embodiment 43 to 45 and further comprising the step,
(c) Growing the cell or organism in the chemical environment selected in step (b).

### Embodiment 47

Cell or organism obtained by the method of embodiment 46.

### Embodiment 48

A method for evolving a metabolic trait of the cell or organism of embodiment 44 comprising the steps,
(a) Determining an evolution chemical environment to evolve the metabolic trait of the cell or organism according to steps (a) and (b) of embodiment 43,
(c) Growing the cell or organism in the evolution chemical environment to evolve the desired trait,
(d) Growing the cell or organism in a target chemical environment to produce at least one desired product by the cell or organism.

### Embodiment 49

Method according to embodiments 1 to 46 and 48, wherein the metabolic trait is the increased generation of at least one aroma compound.

### Embodiment 50

Method according to embodiment 49, wherein the at least one aroma compound is an aromatic aroma compound or branched chain amino acid derived aroma or a precursor thereof.

### Embodiment 51

Method according to embodiment 50, wherein the aromatic aroma compound is phenylethyl acetate,

### Embodiment 52

Method according to embodiments 1 to 46 and 48, wherein the metabolic trait is the increased generation of amino acids that enhance lactic acid bacteria growth.

### Embodiment 53

A computer program element, which, when executed by a processor, is adapted to carry out the method steps according to any of the embodiments 1 to 44.

### Embodiment 54

A computer readable medium, comprising a computer program element according to embodiment 53.

### Embodiment 55

A method for determining a selection chemical environment for screening cells or organisms having a desired metabolic trait comprising the steps of embodiments 43 or 44;

wherein the suitability of the chemical environment to evolve a metabolic trait correlates with the suitability of the chemical environment for screening cells or organisms having a desired metabolic trait.

### Embodiment 56

A method for screening cells or organisms having a desired metabolic trait using a selection chemical environment,
(a) Determining a selection chemical environment according to embodiment 55,
(b) Growing at least two cells or organisms in the selection chemical environment,
(c) Selecting at least one cell or organism that shows higher growth than one or more of the other cells or organisms in the selection environment;
wherein higher growth in the selection environment indicates that the cell or organism contains the desired metabolic trait;

### Embodiment 57

Method according to embodiments 55 or 56, wherein the desired metabolic trait does not provide a fitness benefit in the target chemical environment;

### Embodiment 58

Method according to embodiments 55 to 57, wherein the desired metabolic trait provides a fitness benefit in the selection chemical environment.

1. A method for evaluating the suitability of an evolution chemical environment to evolve a desired metabolic trait of a cell or organism, the method comprising simulation of one or several of functions of fluxes in a metabolic model,
wherein the metabolic model comprises a stoichiometric representation of biochemical reactions and import and export of extracellular compounds,
wherein the metabolic trait comprises a set of targets,
wherein the targets are functions of fluxes.

2. Method according to claim 1, wherein the desired metabolic trait does not provide a fitness benefit in a target chemical environment, where the cell or organism is intended to be used.

3. Method according to claim 1 or 2, wherein the desired metabolic trait provides a fitness benefit in the evolution chemical environment.

4. A method according to any of the preceding claims, wherein simulation of one or several targets relative to growth is performed,
wherein the growth is a function of fluxes through a reaction or reactions that generate biomass components or biomass and wherein a simulation is performed while the growth is constrained to a fixed value or within a range.

5. A method according to any of the preceding claims, wherein simulation is performed while a constraint is set on any of uptakes,
wherein uptakes are functions of fluxes through the reactions representing the import of extracellular compounds.

6. A method according to any of the preceding claims, wherein a simulation is performed while a sum of uptakes is constrained to a fixed value or within a range, wherein optionally a simulation is performed while a sum of uptakes is constrained to an optimal value, and/or wherein a simulation is performed while any of uptakes is constrained to a fixed value or within a range.

7. A method according to any of the preceding claims, wherein up-regulation targets and down-regulation targets are optimized into opposite directions,
wherein the up-regulation targets are those targets for which an increase is desired, and
wherein the down-regulation targets are those targets for which a decrease is desired,
wherein optionally absolute values of up-regulation targets are minimized and absolute values of down-regulation targets are maximized, and/or wherein the number of targets exceeding or falling below at least one threshold is optimized.

8. A method according to any of the preceding claims,
wherein the number of targets relative to a growth exceeding or falling below at least one threshold is simulated and wherein optionally the at least one threshold is determined with respect to functions of fluxes in a reference chemical environment.

9. A method according to any of the preceding claims,
wherein in the simulation at least one inhibited flux is defined, and wherein optionally simulation is performed by constraining to zero fluxes through reactions that are targets of inhibitors or regulatory triggers included in the evolution chemical environment.

10. A method according to any of the preceding claims, further comprising:
determination of a numerical score for the evolution chemical environment,
wherein the score is indicative for a strength of a worst-case selection pressure on the targets in the chemical environment,
wherein the score is indicative for a worst-case coverage of the targets by the selection pressure in the evolution chemical environment, and/or
wherein the score takes into account a number of components in the evolution chemical environment in determining the score of the evolution chemical environment.

11. A method according to any of the preceding claims, wherein simulation of one or several functions of fluxes through the target reactions relative to a growth is performed,
the method comprising the following steps:
(a) Performing a first optimization of the model imposing a plurality of constraints, thereby determining a first optimization result,
wherein a constraint sets a condition defining a growth to a fixed value or in range;
wherein a constraint sets thermodynamic bounds on fluxes in the model;
wherein the first optimization of the model optimizes a sum of uptakes,
(b) Performing a second optimization using the first optimization result, thereby determining a second optimization result,
wherein a constraint sets a growth to a fixed value or in range;
wherein a constraints sets a sum of uptakes to the first optimization result,
wherein the second optimization result is indicative for the suitability of the evolution chemical environment,
wherein the second optimization of the model promotes a condition identified as optimizing a sum of up-regulation targets, and
wherein the second optimization of the model promotes a condition identified as optimizing a sum of down-regulation targets.

12. A method according to the preceding claims, further comprising the step:
(c) Performing a third optimisation of the model, thereby determining a third optimization result,
wherein the number of up-regulation targets which are enhanced with respect to up-regulation targets in a reference chemical environment are optimized, and
wherein the number of down-regulation targets which are suppressed with respect to down-regulation targets in a reference chemical environment are optimized.

13. A method according to claims 9 and 10,
wherein the score is given by the value
$$value = {W}_{c} \cdot \frac{( n - coverage )}{n} + {W}_{s} \cdot \frac{( 1000 \cdot u - strength )}{n} + {W}_{m} \cdot c ,$$
wherein
n denotes the number of targets,
coverage denotes the minimal value of targets obtained in step (c),
u denotes the number of up-regulation targets,
strength denotes the sum of the minimised sum of up-regulation targets and the maximised sum of down-regulation targets obtained in step (b), and
c denotes the number of components in the evolution chemical environment,
W_{c}, Wₛ, and Wₘ denote mathematical weights.

14. A method for determining an evolution chemical environment to evolve a metabolic trait of a cell or organism comprising the following steps:
(a) Evaluating the suitability of at least two chemical environments to evolve a metabolic trait using the method to any one of the preceding claims,
(b) Selecting at least one chemical environment with a suitability to evolve a metabolic trait, which belongs to the chemical environments with the highest potential to evolve a metabolic trait.

15. Method according to claim 14, wherein in step (b) a chemical environment is selected which belongs to the 90%, 80%, 70%, 60%, 50%, 40% 30% 20%, 15%, 10%, 5%, 3%, 2% of chemical environments with the highest potential to evolve a metabolic trait.

16. A method for evolving a metabolic trait of the cell or organism comprising the steps,
(a) Determining an evolution chemical environment to evolve the metabolic trait of the cell or organism according to steps (a) and (b) of claim 12,
(c) Growing the cell or organism in the evolution chemical environment to evolve the desired trait,
(d) Growing the cell or organism in a target chemical environment to produce at least one desired product by the cell or organism.

17. Method for evaluating the suitability of an evolution chemical environment according to claims 1 to 13, method for determining an evolution chemical environment according to claim 14 or 15, method for evolving a metabolic trait according to claim 16, wherein the microorganism is yeast, preferably Saccaromyces sp., more preferably Saccharomyces cerevisiae.

18. Method according to claim 17, wherein the metabolic trait is the increased generation of at least one aroma compound

19. Method according to claim 18, wherein the at least one aroma compound is an aromatic aroma compound or branched chain amino acid derived aroma or a precursor thereof.

20. Method according to claim 19, wherein the aromatic aroma compound is phenylethyl acetate,

21. Method according to claim 17, wherein the metabolic trait is the increased generation of amino acids that enhance lactic acid bacteria growth.

22. Cell or organism obtained by the method of claim 16.

23. A computer readable medium, comprising a computer program element, which, when executed by a processor, is adapted to carry out the method steps according to any of the claims 1 to 11.

24. A method for determining a selection chemical environment for screening cells or organisms having a desired metabolic trait comprising the steps of claim 14 or 15;
wherein the suitability of the chemical environment to evolve a metabolic trait correlates with the suitability of the chemical environment for screening cells or organisms having a desired metabolic trait.

25. A method for screening cells or organisms having a desired metabolic trait using a selection chemical environment,
(a) Determining a selection chemical environment according to claim 24,
(b) Growing at least two cells or organisms in the selection chemical environment,
(c) Selecting at least one cell or organism that shows higher growth than one or more of the other cells or organisms in the selection environment;
wherein higher growth in the selection environment indicates that the cell or organism contains the desired metabolic trait;

26. Method according to claims 24 to 25, wherein the desired metabolic trait does not provide a fitness benefit in the target chemical environment;

27. Method according to claims 24 to 26, wherein the desired metabolic trait provides a fitness benefit in the selection chemical environment.
